(19) United States
(12) Patent Application Publication
(10) Pub. No.: US 2005/0277645 A1
(43) Pub. Date: Dec. 15, 2005

(54) Sleep inducing compounds and methods relating thereto

(75) Inventors: Wilna (Willy) J. Moree, San Diego, CA (US); Graham Beaton, Poway, CA (US); Jinghua Yu, San Marcos, CA (US); Binfeng Li, San Diego, CA (US); Said Zamani-Kord, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA (US)

(21) Appl. No.: 11/097,963

(22) Filed: Apr. 1, 2005

Related U.S. Application Data
(60) Provisional application No. 60/558,642, filed on Apr. 1, 2004.

(51) Int. Cl. A61K 31/4965; A61K 31/426; A61K 31/415

(52) U.S. Cl. 514/252.1; 514/406; 514/365; 544/336; 548/198; 548/373.1

(57) ABSTRACT

Compounds having the following structure: 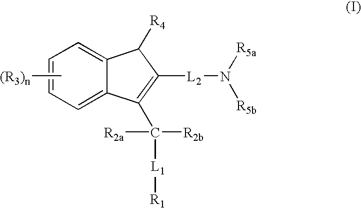
including stereoisomers, prodrugs, and pharmaceutically acceptable salts thereof, wherein R₁, R₂ₐ, R_{2b}, R₃, R₄, R₅ₐ, R_{5b}, L₁, L₂ and n are as defined herein. Pharmaceutical compositions containing one or more compounds of structure (I), as well as methods relating to the use thereof, including methods for treating insomnia, inducing sleep or inducing sedation or hypnosis, are also disclosed.

### CROSS-REFERENCE TO RELATED APPLICATION

This application claims the benefit under 35 U.S.C. § 119(e) of U.S. Provisional Patent Application No. 60/558,642 filed Apr. 1, 2004, which application is incorporated by reference in its entirety.

### BACKGROUND OF THE INVENTION

1. Field of the Invention

This invention relates generally to sleep inducing compounds, as well as to methods for induction of sleep by administration of one or more of such compounds to an animal in need thereof.

2. Description of the Prior Art

Most people experience, at least transiently and often chronically, problems with sleep. Insomnia occurs at all ages with half of all adults in the United States affected at times (The Gallup Organization, Sleep in America: A National Survey of U.S. Adults, The National Sleep Foundation, Washington, D.C., 1995). Insomnia compromises feelings of well-being and judgment and performance at tasks requiring alertness (Gillin, Postgrad Med. 1992; 92: 157-160). Significantly, inadequate sleep correlates with increased morbidity and mortality (Zammit et al, Sleep 1999;22: S379-85).

In a clinical setting, insomnia may be classified as transient, short-term, or chronic, with durations of a few days, a few weeks, or long-term, respectively (Chessor, Sleep 2000;22: 237-41). Common etiologies for transient insomnia include acute illness, social stress, jet lag, and work shift changes. Short-term insomnias can be caused by grief, stress, and substance exposure. Chronic insomnias can be associated with underlying disease, depression, psychophysiologic conditions, chronic stress, bereavement, substance exposure, and a variety of primary sleep disorders including sleep apnea, periodic limb movement disorder, restless leg syndrome, narcolepsy and hypersomnia. The primary task of the physician is to identify the specific etiology of the insomnia and prescribe a causally specific therapeutic intervention (Pary et al, Postgrad Med. 1996; 100: 195-210).

The categories of drugs used as sedative-hypnotics in the United States (Wang et al, Drug Disposition and Pharmacokinetics 2003; 37: 10-29) include barbiturates, the benzodiazepine hypnotics, benzodiazepine nonhypnotics, benzodiazepine receptor agonists, antidepressants, antipsychotics, miscellaneous compounds including chloral hydrate, and the antihistamines.

With regard to the antihistamines, histamine enjoys a variety of important chemical messenger roles, having activity toward at least four histamine receptors (i.e., H₁-H₄) and regulatory function in the nervous, gastrointestinal and immune systems. The antihistamines are reversible competitive ligands of the histamine H₁ receptor, and have been categorized over the years as first-, second-, or third-generation classes differentiated by chemical structure and refinement of action. Specifically, first-generation antihistamines such as diphenhydramine, chlorpheniramine, clemastine, hydroxyzine and triprolidine provide H₁ receptor blockade, but have significant side effects including sedation, CNS dysfunction due to leakage into the CNS, and anticholinergic adverse effects. In response, the second-generation or so-called “nonsedating” antihistamines, including astemizole, terfenadine, loratadine, cetirizine and fexofenadine, were developed. These compounds have reduced CNS impact and additional antiallergic properties, including inhibition of mast cell degranulation. Desloratadine, a metabolite of loratadine, has been categorized as a third-generation antihistamine (McClellan & Jarvis, Drugs 2001; 61: 789-796), and has direct effects on inflammatory mediators such as inhibition of intracellular adhesion molecule-1 (ICAM-1) expression by nasal epithelium. The importance of histamine in sleep regulation is evidenced by the hypnotic effects of certain histamine receptor ligands (Mignot et al., Nature Neuroscience Supplement; 5; 1071-1075), particularly the older generation molecules.

While significant advances have been made in the field of sleep initiation and prolongation, there continues to be a need in the art for compounds that are effective as sedative and hypnotic agents, especially for compounds with clinical application to the treatment of insomnia. In particular, there remains a need for compounds having improved selectivity, a quicker onset of action, a shorter half-life and/or the ability to penetrate the CNS. The present invention fulfills these needs and other needs, and provides further related advantages.

### BRIEF SUMMARY OF THE INVENTION

In brief, this invention is directed to compounds that have utility over a wide range of therapeutic applications, particularly in the context of inducing sleep, and which compounds have the following general structure (I): 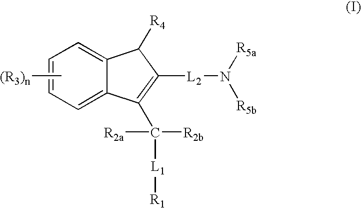
including stereoisomers, prodrugs and pharmaceutically acceptable salts thereof, wherein R₁, R₂ₐ, R_{2b}, R₃, R₄, R₅ₐ, R_{5b}, L₁, L₂ and n are as defined below.

The compounds of this invention may generally be used to treat a variety of disorders and/or illnesses, particularly those that benefit from inhibition of one or more histamine receptors. Accordingly, in one embodiment, methods for treating a condition or disorder are disclosed, the treatment of which can be effected or facilitated by antagonizing a histamine receptor. In another embodiment, methods are disclosed for treating sleep disorders, including insomnia, as well as for inducing sleep, sedation and/or hypnosis generally.

The methods of this invention generally involve administering an effective amount of one or more compounds of this invention, typically in the form of a pharmaceutical composition, to an animal (also referred to here as a “patient”, including a human) in need thereof. Accordingly, in still another embodiment, compositions are disclosed containing one or more compounds of this invention in combination with a pharmaceutically acceptable carrier and/or diluent.

These and other aspects of the invention will be apparent upon reference to the following detailed description. To that end, various references are set forth herein which describe in more detail certain procedures, compounds and/or compositions, and are hereby incorporated by reference in their entirety.

### DETAILED DESCRIPTION OF THE INVENTION

As noted above, this invention is generally directed to compounds that have utility over a wide range of therapeutic applications, particularly in the context of sleep induction, and more particularly for treatment of insomnia.

Accordingly, in one embodiment, this invention is directed to a method for treatment of a sleep disorder comprising administering to a patient in need thereof an effective amount of a compound having the following structure (I): 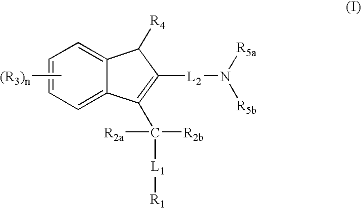
including stereoisomers, prodrugs and pharmaceutically acceptable salts thereof,

wherein:
R₁ is R₁ₐ, R_{1b}, —OR, —CN, —C(═O)R, —OC(═O)R or —C(═O)OR, wherein R is alkyl or substituted alkyl;
R₁ₐ is heterocycle or substituted heterocycle, with the proviso that R₁ₐ is not pyridinyl or substituted pyridinyl;
R_{1b} is bicyclic carbocycle or substituted bicyclic carbocycle;
L₁ is a bond or L₂;
L₂ is alkanediyl or substituted alkanediyl;
R₂ₐ and R_{2b} are the same or different and are independently hydrogen, alkyl or substituted alkyl;
R₃ is, at each occurrence, the same or different and independently alkyl, —OR, —SR, —CN, —CF₃ or halogen, wherein R is alkyl or substituted alkyl;
R₄ is hydrogen or alkyl;
R₅ₐ and R_{5b} are the same or different and independently hydrogen, alkyl or substituted alkyl, or R₅ₐ and R_{5b} together with the nitrogen to which they are attached form a heterocycle or substituted heterocycle; and
n is 0, 1 or 2 and represents the number of R₃ groups.

Without intending to be bound by theory, it is believed that the compounds of this invention function as ligands to one or more histamine receptors, and are thereby useful in the treatment of a variety of conditions or diseases associated therewith. In this manner, the compounds alter or regulate the activity of a histamine receptor, thereby providing a treatment for a condition or disease associated with that receptor. Thus, compounds of this invention may have utility over a broad range of therapeutic applications, and may be used to treat disorders or illnesses, including (but not limited to) sleep disorders. Compounds of this invention may be advantageous as sedative hypnotics as they show one or more enhancements over previously known antihistamines. Some advantages of compounds of the present invention may include an enhanced selectivity profile for H₁ receptor relative to other G-protein coupled receptors and other proteins in comparison to other known sedating H₁ ligands. Effects on sleep processes are therefore more specific. Compounds of the present invention may also show reduced inhibition of cytochrome P₄₅₀ (CYP) enzymes that potentiate drug interactions as well as other proteins associated with the safety of pharmaceuticals such as the human ether a go-go (hERG) channel. These compounds may also show favorable characteristics relative to other known antihistamines, including (but not limited to) improved efficacy, improved quality of sleep, lack of peripheral side effects and optimal pharmacokinetics for use as a sedative. Accordingly, the methods of this invention include, in addition to treatment of a sleep disorders as noted above, treatment of insomnia, as well as for inducing sleep, sedation and/or hypnosis, by administration of an effective amount of a compound of structure (I) as disclosed above to a patient in need thereof.

In another embodiment, compounds are disclosed having the following structure (II): 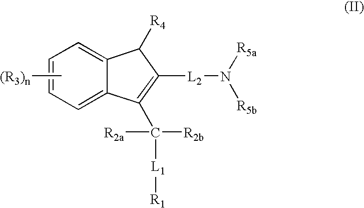
including stereoisomers, prodrugs and pharmaceutically acceptable salts thereof,

wherein:
R₁ is R₁ₐ, R_{1b}, —OR, —CN, —C(═O)R, —OC(═O)R or —C(═O)OR, wherein R is alkyl or substituted alkyl;
R₁ₐ is heterocycle or substituted heterocycle, with the proviso that R₁ₐ is not pyridinyl or substituted pyridinyl;
R_{1b} is bicyclic carbocycle or substituted bicyclic carbocycle;
L₁ is a bond or L₂;
L₂ is alkanediyl or substituted alkanediyl;
R₂ₐ and R_{2b} are the same or different and are independently hydrogen, alkyl or substituted alkyl, with the proviso that R₂ₐ and R_{2b} are not both hydrogen;
R₃ is, at each occurrence, the same or different and independently alkyl, —OR, —SR, —CN, —CF₃ or halogen, wherein R is alkyl or substituted alkyl;
R₄ is hydrogen or alkyl;
R₅ₐ and R_{5b} are the same or different and independently hydrogen, alkyl or substituted alkyl, or R₅ₐ and R_{5b} together with the nitrogen to which they are attached form a heterocycle or substituted heterocycle; and
n is 0, 1 or 2 and represents the number of R₃ groups.

In still another embodiment, compounds are disclosed having the following structure (III): 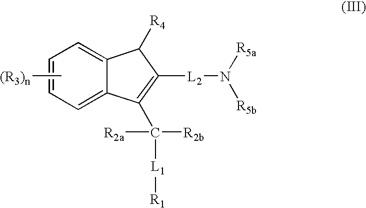
including stereoisomers, prodrugs and pharmaceutically acceptable salts thereof,

wherein:
R₁ is R₁ₐ, wherein R₁ₐ is pyrazinyl, substituted pyrazinyl, pyridazinyl, substituted pyridazinyl, triazinyl, or substituted triazinyl;
L₁ is a bond or L₂;
L₂ is alkanediyl or substituted alkanediyl;
R₂ₐ and R_{2b} are both hydrogen;
R₃ is, at each occurrence, the same or different and independently alkyl, —OR, —SR, —CN, —CF₃ or halogen, wherein R is alkyl or substituted alkyl;
R₄ is hydrogen or alkyl;
R₅ₐ and R_{5b} are the same or different and independently hydrogen, alkyl or substituted alkyl, or R₅ₐ and R_{5b} together with the nitrogen to which they are attached form a heterocycle or substituted heterocycle; and
n is 0, 1 or 2 and represents the number of R₃ groups.

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclopropyl, —CH₂-cyclobutyl, —CH₂-cyclopentyl, —CH₂-cyclohexyl, and the like; while unsaturated cyclic alkyls include cyclopentenyl and cyclohexenyl, and the like. Cyclic alkyls, also referred to as “homocyclic rings,” and include di- and poly-homocyclic rings such as decalin and adamantyl. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like.

“Bicyclic carbocycle” means a 7- to 13-membered carbon ring system containing no ring heteroatoms and having two carbon rings which share at least two carbon atoms, and which may be saturated, partially unsaturated or aromatic, such as naphthyl, indanyl, 5H-benzocycloheptene, tetrahydronaphthyl, and the like.

“Alkanediyl” means a divalent alkyl from which two hydrogen atoms are taken from the same carbon atom or from different carbon atoms, such as —CH₂—, —CH₂CH₂—, —CH₂CH₂CH₂—, —CH(CH₃)CH₂—, -cyclopentane-, -cyclohexane-, -cycloheptane-, and the like.

“Aryl” means an aromatic carbocyclic moiety such as phenyl or naphthyl.

“Arylalkyl” means an alkyl having at least one alkyl hydrogen atoms replaced with an aryl moiety, such as benzyl (i.e., —CH₂-phenyl), —CH₂-(1- or 2-naphthyl), —(CH₂)₂-phenyl, —(CH₂)₃-phenyl, —CH(phenyl)₂, and the like.

“Heteroaryl” means an aromatic heterocycle ring of 5- to 10-members and having at least one heteroatom selected from nitrogen, oxygen and sulfur, and containing at least 1 carbon atom, including both mono- and bicyclic ring systems. Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂-pyridinyl, —CH₂-pyrimidinyl, and the like.

“Heterocycle” (also referred to herein as a “heterocycle ring”) means a 5- to 7-membered monocyclic, or 7- to 14-membered polycyclic, heterocycle ring which is either saturated, unsaturated or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring as well as tricyclic (and higher) heterocyclic rings. The heterocycle may be attached via any heteroatom or carbon atom. Heterocycles include heteroaryls as defined above. Thus, in addition to the aromatic heteroaryls listed above, heterocycles also include (but are not limited to) morpholinyl, pyrrolidinonyl, pyrrolidinyl, piperizinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle, such as —CH₂-morpholinyl, and the like.

“Halogen” means fluoro, chloro, bromo and iodo.

“Haloalkyl” means an alkyl having at least one hydrogen atom replaced with halogen, such as trifluoromethyl and the like. Haloalkyl is a specific embodiment of substituted alkyl, wherein alkyl is substituted with one or more halogen atoms.

“Hydroxyalkyl” means an alkyl having at least one hydrogen atom replaced.

“Alkoxy” means an alkyl moiety attached through an oxygen bridge (i.e., —O-alkyl), such as —O-methyl, —O-ethyl, and the like.

“Thioalkyl” means an alkyl moiety attached through a sulfur bridge (i.e., —S-alkyl) such as —S-methyl, —S-ethyl, and the like.

“Aryloxy” means an aromatic carbocyclic moiety such as phenyl or naphthyl attached through an oxygen bridge (i.e., —O-aryl) such as —O-phenyl, —O-naphthyl, and the like.

Lastly, the term “substituted” as used herein means any of the above groups (i.e., alkyl, alkanediyl, bicyclic carbocycle, aryl, arylalkyl, heteroaryl, heteroarylalkyl, heterocycle or heterocyclealkyl) wherein at least one hydrogen atom is replaced with a substituent. In the case of an oxo substituent (“═O”) two hydrogen atoms are replaced. “Substituents” within the context of this invention include halogen, hydroxy, oxo, cyano, nitro, amino, alkylamino, dialkylamino, alkyl, alkoxy, thioalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b}—NRₐSO₂R_{b}, —ORₐ, —C(═O)Rₐ—C(═O)ORₐ, —C(═O)NRₐR_{b}, —OC(═O)NRₐR_{b}, —SH, —SRₐ, —SORₐ, —S(═O)₂Rₐ, —OS(═O)₂Rₐ, —S(═O)₂ORₐ, wherein Rₐ and R_{b} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

In more specific embodiments of structures (I), (II) and (III), L₁ is a bond (i.e., a direct bond between R₁ and the carbon atom bearing R₂ₐ and R_{2b}), and compounds of this invention have the following structure (IV). In an alternative embodiment, L₁ is alkanediyl or substituted alkanediyl, such as —CH₂— as represented in structure (V).

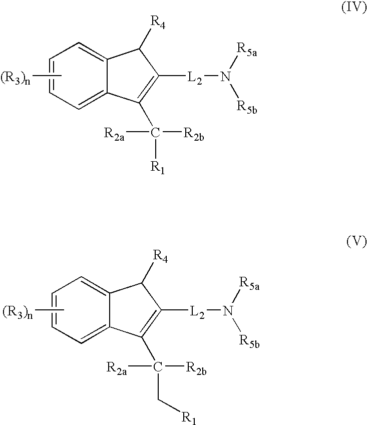

As noted above, R₂ₐ and R_{2b} are the same or different and are independently hydrogen, alkyl or substituted alkyl. When R₂ₐ and R_{2b} are different, the carbon atom to which R₂ₐ and R_{2b} are bonded is a chiral center (i.e., a carbon atom to which four different groups are attached). Accordingly, in more specific embodiments of structures (I) and (II), R₂ₐ and R_{2b} are different, and the carbon to which these groups are bonded is a chiral center, as represented in the following structure (VI) by the asteric symbol “*” on the chiral carbon atom. For example, representative R₂ₐ and R_{2b} combinations include the following embodiments: R₂ₐ is hydrogen and R_{2b} is either alkyl or substituted alkyl; R₂ₐ is alkyl and R_{2b} is substituted alkyl; R₂ₐ is alkyl and R_{2b} is a different alkyl; R₂ₐ is substituted alkyl and R_{2b} is a different substituted alkyl.

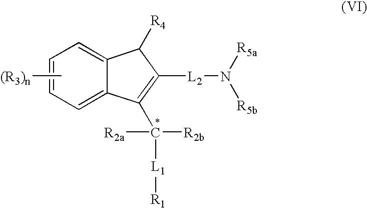

In a more specific embodiment of structure (VI), R₁ is R₁ₐ, and R₁ₐ is heterocycle or substituted heterocycle.

In more specific embodiment of structures (I) and (III), R₂ₐ and R_{2b} are the same, and the carbon to which these groups are bonded is not a chiral center, as represented in the following structure (VII) by the symbol “\hat{ }” on the non-chiral carbon atom. For example, representative R₂ₐ and R_{2b} combinations include the following embodiments: R₂ₐ and R_{2b} are both hydrogen (i.e., structure (III)); R₂ₐ and R_{2b} are both the same alkyl; or R₂ₐ and R_{2b} are both the same substituted alkyl.

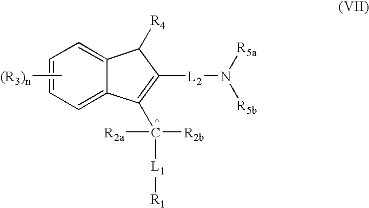

In a more specific aspect of structure (VII), R₁ is R₁ₐ, R₁ₐ is pyrazinyl or substituted pyrazinyl, and the compounds have the following structure (VIII-1) or (VIII-2), respectively:

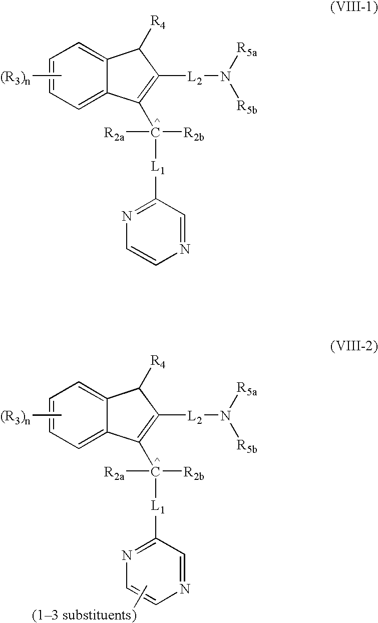

When L₁ of structure (VIII-1) or (VIII-2) is a bond, the compounds of this invention have the following structures (IX-1) or (IX-2), respectively:

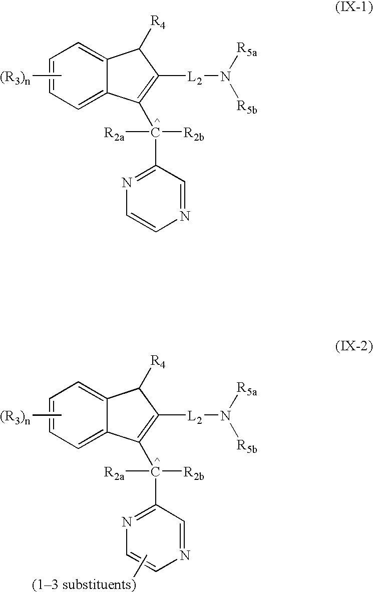

In more specific embodiments of structures (I), (II) and (III), L₂ is alkanediyl such as —CH₂— or —CH₂CH₂—, and compounds of this invention have the following structures (X-1) or (X-2), respectively:

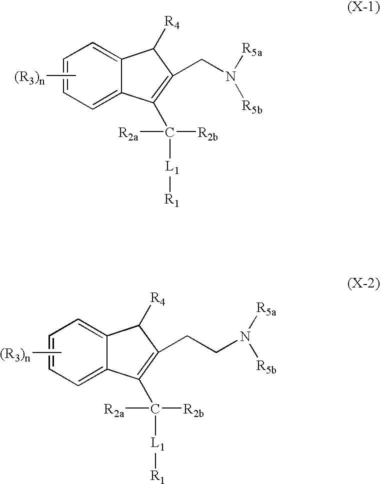

In another more specific embodiment of structures (I), (II) and (III), n is 0 and R₃ is not present (i.e., n is 0). In another embodiment, n is 1 and R₃ is alkyl, —OR, —SR, CN, —CF₃ or halogen, wherein R is alkyl or substituted alkyl, or n is 2 and R₃ is, at each occurrence, the same or different and independently alkyl, —OR, —SR, —CN, —CF₃ or halogen, wherein R is alkyl or substituted alkyl.

In another more specific embodiment of structures (I), (II) and (III), R₄ is hydrogen, and in another embodiment R₄ is alkyl including (but not limited to) lower alkyl such as methyl, ethyl and the like.

In another more specific embodiment of structures (I), (II) and (III), R₅ₐ and R_{5b} are the same or different and independently hydrogen, alkyl or substituted alkyl or, alternatively, R₅ₐ and R_{5b} together with the nitrogen to which they are attached form a heterocycle or substituted heterocycle (such as a heterocyclic ring which is optionally substituted with alkyl or substituted alkyl).

In still further and more specific embodiments of structures (I) and (II), L₁ is a bond, R₂ₐ is hydrogen, R_{2b} is methyl, and the compounds of this invention have the following structure (XI):

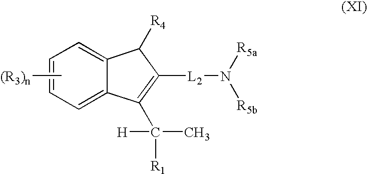

In more specific embodiments of structure (XI), R₁ is R₁ₐ, and R₁ₐ is pyrazinyl or 5-methyl-1,3,4-oxadiazolyl as represented by the following structures (XII-1) and (XII-2), respectively:

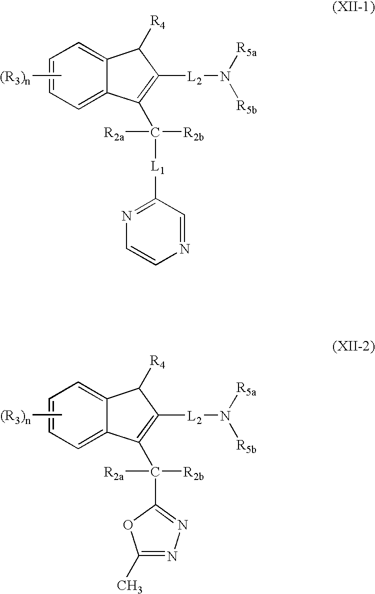

In another more specific embodiment of structure (XI), R₁ is —C(═O)OR, such as —C(═O)O(tert-butyl) as represented in structure (XIII):

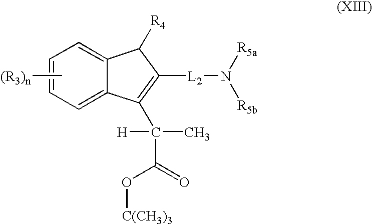

In still further and more specific embodiments of structures (I) and (III), R₁ is —OCH₂CH₃, L₁ is a —CH₂—, L₂ is —CH₂CH₂—, R₂ₐ is hydrogen, R_{2b} is methyl, and the compounds of this invention have the following structure (XIV):

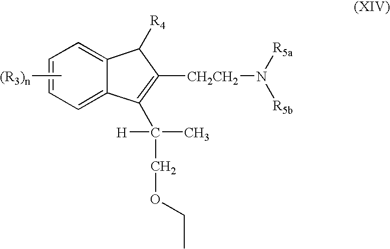

In more specific embodiment of structures (XI), (XII-1), (XII-2), (XIII) and (XIV), n is 0 and/or R₄ is hydrogen.

Representative compounds of the present invention include the following and their enantiomers:

(2-Fluoro-ethyl)-methyl-{2-[3-(1-pyrazin-2-yl-ethyl)-1H-inden-2-yl]-ethyl}-amine;

(2-Fluoro-ethyl)-methyl-{2-[6-methyl-3-(1-pyrazin-2-yl-ethyl)-1H-inden-2-yl]-ethyl}-amine;

(2-Fluoro-ethyl)-{2-[6-methoxy-3-(1-pyrazin-2-yl-ethyl)-1H-inden-2-yl]-ethyl}-methyl-amine;

Dimethyl-{2-[3-(1-pyridazin-3-yl-ethyl)-1H-inden-2-yl]-ethyl}-amine;

Dimethyl-{2-[6-methyl-3-(1-pyridazin-3-yl-ethyl)-1H-inden-2-yl]-ethyl}-amine;

{2-[6-Methoxy-3-(1-pyridazin-3-yl-ethyl)-1H-inden-2-yl]-ethyl}-dimethyl-amine;

{2-[6-Fluoro-3-(1-pyridazin-3-yl-ethyl)-1H-inden-2-yl]-ethyl}-dimethyl-amine;

1-{2-[3-(1-Pyrazin-2-yl-ethyl)-1H-inden-2-yl]-ethyl}-azetidin-3-ol;

2-(1-{2-[2-(3-Fluoro-azetidin-1-yl)-ethyl]-3H-inden-1-yl}-ethyl)-pyrazine

1-{2-[3-(1-Pyrazin-2-yl-ethyl)-1H-inden-2-yl]-ethyl}-pyrrolidin-3-ol;

2-(1-{2-[2-(3-Fluoro-pyrrolidin-1-yl)-ethyl]-3H-inden-1-yl}-ethyl)-pyrazine;

1-(2-{6-Fluoro-3-[1-(3-methoxy-pyrazin-2-yl)-ethyl]-1H-inden-2-yl}-ethyl)-pyrrolidin-3-ol;

1-(2-{6-Fluoro-3-[1-(3-methoxy-pyrazin-2-yl)-ethyl]-1H-inden-2-yl}-ethyl)-azetidin-3-ol;

2-(2-Dimethylamino-ethyl)-1-[1-(3-methoxy-pyrazin-2-yl)-ethyl]-3H-indene-5-carbonitrile;

2-(2-Dimethylamino-ethyl)-1-(1-pyrazin-2-yl-ethyl)-3H-indene-5-carbonitrile;

2-(2-Dimethylamino-ethyl)-1-pyridazin-3-ylmethyl-3H-indene-5-carbonitrile;

2-{2-[2-(2,5-Dihydro-pyrrol-1-yl)-ethyl]-3H-inden-1-ylmethyl}-pyrazine;

3-{2-[2-(2,5-Dihydro-pyrrol-1-yl)-ethyl]-3H-inden-1-ylmethyl}-pyridazine;

3-{2-[2-(2,5-Dihydro-pyrrol-1-yl)-ethyl]-5-methyl-3H-inden-1-ylmethyl}-pyridazine;

3-{2-[2-(2,5-Dihydro-pyrrol-1-yl)-ethyl]-5-fluoro-3H-inden-1-ylmethyl}-pyridazine;

3-{2-[2-(2,5-Dihydro-pyrrol-1-yl)-ethyl]-5-methoxy-3H-inden-1-ylmethyl}-pyridazine;

2-{2-[2-(2,5-Dihydro-pyrrol-1-yl)-ethyl]-3H-inden-1-ylmethyl}-3-methoxy-pyrazine;

2-{2-[2-(2,5-Dihydro-pyrrol-1-yl)-ethyl]-5-methyl-3H-inden-1-ylmethyl}-3-methoxy-pyrazine;

2-{2-[2-(2,5-Dihydro-pyrrol-1-yl)-ethyl]-5-fluoro-3H-inden-1-ylmethyl}-3-methoxy-pyrazine;

2-{2-[2-(2,5-Dihydro-pyrrol-1-yl)-ethyl]-5-methoxy-3H-inden-1-ylmethyl}-3-methoxy-pyrazine;

2-(1-{2-[2-(2,5-Dihydro-pyrrol-1-yl)-ethyl]-3H-inden-1-yl}-ethyl)-pyrazine;

2-(1-{2-[2-(2,5-Dihydro-pyrrol-1-yl)-ethyl]-5-methyl-3H-inden-1-yl}-ethyl)-pyrazine;

2-(1-{2-[2-(2,5-Dihydro-pyrrol-1-yl)-ethyl]-5-fluoro-3H-inden-1-yl}-ethyl)-pyrazine;

2-(1-{2-[2-(2,5-Dihydro-pyrrol-1-yl)-ethyl]-5-methoxy-3H-inden-1-yl}-ethyl)-pyrazine;

2-(1-{2-[2-(2,5-Dihydro-pyrrol-1-yl)-ethyl]-3H-inden-1-yl}-ethyl)-3-methoxy-pyrazine;

2-(1-{2-[2-(2,5-Dihydro-pyrrol-1-yl)-ethyl]-5-methyl-3H-inden-1-yl}-ethyl)-3-methoxy-pyrazine;

2-(1-{2-[2-(2,5-Dihydro-pyrrol-1-yl)-ethyl]-5-fluoro-3H-inden-1-yl}-ethyl)-3-methoxy-pyrazine;

2-(1-{2-[2-(2,5-Dihydro-pyrrol-1-yl)-ethyl]-5-methoxy-3H-inden-1-yl}-ethyl)-3-methoxy-pyrazine;

3-(1-{2-[2-(2,5-Dihydro-pyrrol-1-yl)-ethyl]-3H-inden-1-yl}-ethyl)-pyridazine;

3-(1-{2-[2-(2,5-Dihydro-pyrrol-1-yl)-ethyl]-5-methyl-3H-inden-1-yl}-ethyl)-pyridazine;

2-{1-[2-(2-Azetidin-1-yl-ethyl)-5-fluoro-3H-inden-1-yl]-ethyl}-3-methoxy-pyrazine;

3-{1-[2-(2-Azetidin-1-yl-ethyl)-5-fluoro-3H-inden-1-yl]-ethyl}-pyridazine;

3-{2-[2-(3-Fluoro-pyrrolidin-1-yl)-ethyl]-3H-inden-1-ylmethyl}-pyridazine;

3-{2-[2-(3-Fluoro-pyrrolidin-1-yl)-ethyl]-5-methyl-3H-inden-1-ylmethyl}-pyridazine;

3-{5-Fluoro-2-[2-(3-fluoro-pyrrolidin-1-yl)-ethyl]-3H-inden-1-ylmethyl}-pyridazine;

3-{2-[2-(3-Fluoro-pyrrolidin-1-yl)-ethyl]-5-methoxy-3H-inden-1-ylmethyl}-pyridazine;

1-[2-(3-Pyrazin-2-ylmethyl-1H-inden-2-yl)-ethyl]-pyrrolidin-3-ol;

1-{2-[6-Methyl-3-(1-pyrazin-2-yl-ethyl)-1H-inden-2-yl]-ethyl}-pyrrolidin-3-ol;

2-(1-{2-[2-(3-Fluoro-pyrrolidin-1-yl)-ethyl]-5-methyl-3H-inden-1-yl}-ethyl)-pyrazine;

2-(1-{2-[2-(3-Fluoro-pyrrolidin-1-yl)-ethyl]-5-methoxy-3H-inden-1-yl}-ethyl)-pyrazine;

1-{2-[6-Methoxy-3-(1-pyrazin-2-yl-ethyl)-1H-inden-2-yl]-ethyl}-pyrrolidin-3-ol;

1-{2-[6-Fluoro-3-(1-pyrazin-2-yl-ethyl)-1H-inden-2-yl]-ethyl}-pyrrolidin-3-ol;

1-{2-[3-(1-Pyridazin-3-yl-ethyl)-1H-inden-2-yl]-ethyl}-pyrrolidin-3-ol;

1-{2-[6-Methoxy-3-(1-pyridazin-3-yl-ethyl)-1H-inden-2-yl]-ethyl}-pyrrolidin-3-ol;

1-{2-[6-Fluoro-3-(1-pyridazin-3-yl-ethyl)-1H-inden-2-yl]-ethyl}-pyrrolidin-3-ol;

3-(1-{2-[2-(3-Fluoro-pyrrolidin-1-yl)-ethyl]-3H-inden-1-yl}-ethyl)-pyridazine;

3-(1-{2-[2-(3-Fluoro-pyrrolidin-1-yl)-ethyl]-5-methyl-3H-inden-1-yl}-ethyl)-pyridazine;

3-(1-{2-[2-(3-Fluoro-pyrrolidin-1-yl)-ethyl]-5-methoxy-3H-inden-1-yl}-ethyl)-pyridazine;

3-(1-{5-Fluoro-2-[2-(3-fluoro-pyrrolidin-1-yl)-ethyl]-3H-inden-1-yl}-ethyl)-pyridazine;

1-(2-{3-[1-(3-Methoxy-pyrazin-2-yl)-ethyl]-1H-inden-2-yl}-ethyl)-pyrrolidin-3-ol;

1-(2-{3-[1-(3-Methoxy-pyrazin-2-yl)-ethyl]-6-methyl-1H-inden-2-yl}-ethyl)-pyrrolidin-3-ol;

1-(2-{6-Methoxy-3-[1-(3-methoxy-pyrazin-2-yl)-ethyl]-1H-inden-2-yl}-ethyl)-pyrrolidin-3-ol;

2-(1-{2-[2-(3-Fluoro-pyrrolidin-1-yl)-ethyl]-3H-inden-1-yl}-ethyl)-3-methoxy-pyrazine;

2-(1-{2-[2-(3-Fluoro-pyrrolidin-1-yl)-ethyl]-5-methyl-3H-inden-1-yl}-ethyl)-3-methoxy-pyrazine;

2-(1-{2-[2-(3-Fluoro-pyrrolidin-1-yl)-ethyl]-5-methoxy-3H-inden-1-yl}-ethyl)-3-methoxy-pyrazine;

2-(1-{5-Fluoro-2-[2-(3-fluoro-pyrrolidin-1-yl)-ethyl]-3H-inden-1-yl}-ethyl)-3-methoxy-pyrazine;

2-{2-[2-(3-Fluoro-pyrrolidin-1-yl)-ethyl]-3H-inden-1-ylmethyl}-3-methoxy-pyrazine;

2-{2-[2-(3-Fluoro-pyrrolidin-1-yl)-ethyl]-5-methyl-3H-inden-1-ylmethyl}-3-methoxy-pyrazine;

2-{2-[2-(3-Fluoro-pyrrolidin-1-yl)-ethyl]-5-methoxy-3H-inden-1-ylmethyl}-3-methoxy-pyrazine;

2-{5-Fluoro-2-[2-(3-fluoro-pyrrolidin-1-yl)-ethyl]-3H-inden-1-ylmethyl}-3-methoxy-pyrazine;

3-{1-[2-(2-Dimethylamino-ethyl)-5-methoxy-3H-inden-1-yl]-ethyl}-pyrazin-2-ol;

2-({2-[3-(2-Methoxy-1-methyl-ethyl)-1H-inden-2-yl]-ethyl}-methyl-amino)-ethanol;

Dimethyl-{2-[3-(1-pyrazol-1-yl-ethyl)-1H-inden-2-yl]-ethyl}-amine;

Dimethyl-{2-[6-methyl-3-(1-pyrazol-1-yl-ethyl)-1H-inden-2-yl]-ethyl}-amine;

{2-[6-Methoxy-3-(1-pyrazol-1-yl-ethyl)-1H-inden-2-yl]-ethyl}-dimethyl-amine;

{2-[6-Fluoro-3-(1-pyrazol-1-yl-ethyl)-1H-inden-2-yl]-ethyl}-dimethyl-amine;

Dimethyl-{2-[3-(1-[1,2,3]triazol-1-yl-ethyl)-1H-inden-2-yl]-ethyl}-amine;

Dimethyl-{2-[6-methyl-3-(1-[1,2,3]triazol-1-yl-ethyl)-1H-inden-2-yl]-ethyl}-amine;

{2-[6-Fluoro-3-(1-[1,2,3]triazol-1-yl-ethyl)-1H-inden-2-yl]-ethyl}-dimethyl-amine;

{2-[6-Methoxy-3-(1-[1,2,3]triazol-1-yl-ethyl)-1H-inden-2-yl]-ethyl}-dimethyl-amine;

2-( {2-[6-Methoxy-3-(1-thiazol-2-yl-ethyl)-1H-inden-2-yl]-ethyl}-methyl-amino)-ethanol;

1-{2-[6-Methoxy-3-(1-thiazol-2-yl-ethyl)-1H-inden-2-yl]-ethyl}-pyrrolidin-3-ol;

1-{2-[6-Methoxy-3-(1-thiazol-2-yl-ethyl)-1H-inden-2-yl]-ethyl}-azetidin-3-ol;

(2-Fluoro-ethyl)-{2-[6-methoxy-3-(1-thiazol-2-yl-ethyl)-1H-inden-2-yl]-ethyl}-methyl-amine;

2-(Methyl-{2-[3-(1-thiazol-2-yl-ethyl)-1H-inden-2-yl]-ethyl}-amino)-ethanol;

(2-Fluoro-ethyl)-methyl-{2-[3-(1-thiazol-2-yl-ethyl)-1H-inden-2-yl]-ethyl}-amine;

1-{2-[3-(1-Thiazol-2-yl-ethyl)-1H-inden-2-yl]-ethyl}-pyrrolidin-3-ol;

2-(1-{2-[2-(3-Fluoro-pyrrolidin-1-yl)-ethyl]-3H-inden-1-yl}-ethyl)-thiazole;

1-{2-[3-(1-Thiazol-2-yl-ethyl)-1H-inden-2-yl]-ethyl}-azetidin-3-ol;

2-(1-{2-[2-(3-Fluoro-azetidin-1-yl)-ethyl]-3H-inden-1-yl}-ethyl)-thiazole;

2-(Methyl-{2-[3-(1-pyrazin-2-yl-ethyl)-1H-inden-2-yl]-ethyl}-amino)-ethanol;

2-({2-[6-Fluoro-3-(1-pyrazin-2-yl-ethyl)-1H-inden-2-yl]-ethyl}-methyl-amino)-ethanol;

(2-Fluoro-ethyl)-{2-[6-fluoro-3-(1-pyrazin-2-yl-ethyl)-1H-inden-2-yl]-ethyl}-methyl-amine;

(2-Fluoro-ethyl)-(2-{6-fluoro-3-[1-(3-methoxy-pyrazin-2-yl)-ethyl]-1H-inden-2-yl}-ethyl)-methyl-amine;

2-[(2-{6-Fluoro-3-[1-(3-methoxy-pyrazin-2-yl)-ethyl]-1H-inden-2-yl}-ethyl)-methyl-amino]-ethanol;

2-[(2-{3-[1-(3-Methoxy-pyrazin-2-yl)-ethyl]-1H-inden-2-yl}-ethyl)-methyl-amino]-ethanol;

(2-Fluoro-ethyl)-(2-{3-[1-(3-methoxy-pyrazin-2-yl)-ethyl]-1H-inden-2-yl}-ethyl)-methyl-amine; and

2-{2-[2-(3-Fluoro-pyrrolidin-1-yl)-ethyl]-3H-inden-1-ylmethyl}-pyrazine.

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structures (I), (II) and (III) is intended to encompass any and all acceptable salt forms.

In addition, prodrugs are also included within the context of this invention. Prodrugs are any covalently bonded carriers that release a compound of structures (I), (II) and (III) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structures (I), (II) and (III). Further, in the case of a carboxylic acid (—COOH), esters may be employed, such as methyl esters, ethyl esters, and the like.

With regard to stereoisomers, the compounds of structures (I), (II) and (III) may have chiral centers and may occur as racemates, racemic mixtures and as individual enantiomers or diastereomers. All such isomeric forms are included within the present invention, including mixtures thereof. Compounds of structures (I), (II) and (III) may also possess axial chirality which may result in atropisomers. Furthermore, some of the crystalline forms of the compounds of structures (I), (II) and (III) may exist as polymorphs, which are included in the present invention. In addition, some of the compounds of structures (I), (II) and (III) may also form solvates with water or other organic solvents. Such solvates are similarly included within the scope of this invention.

The compounds of this invention may be prepared by known organic synthesis techniques, including the methods of the following Reaction Schemes 1-8, as well as by the more detailed methods disclosed in the Examples.

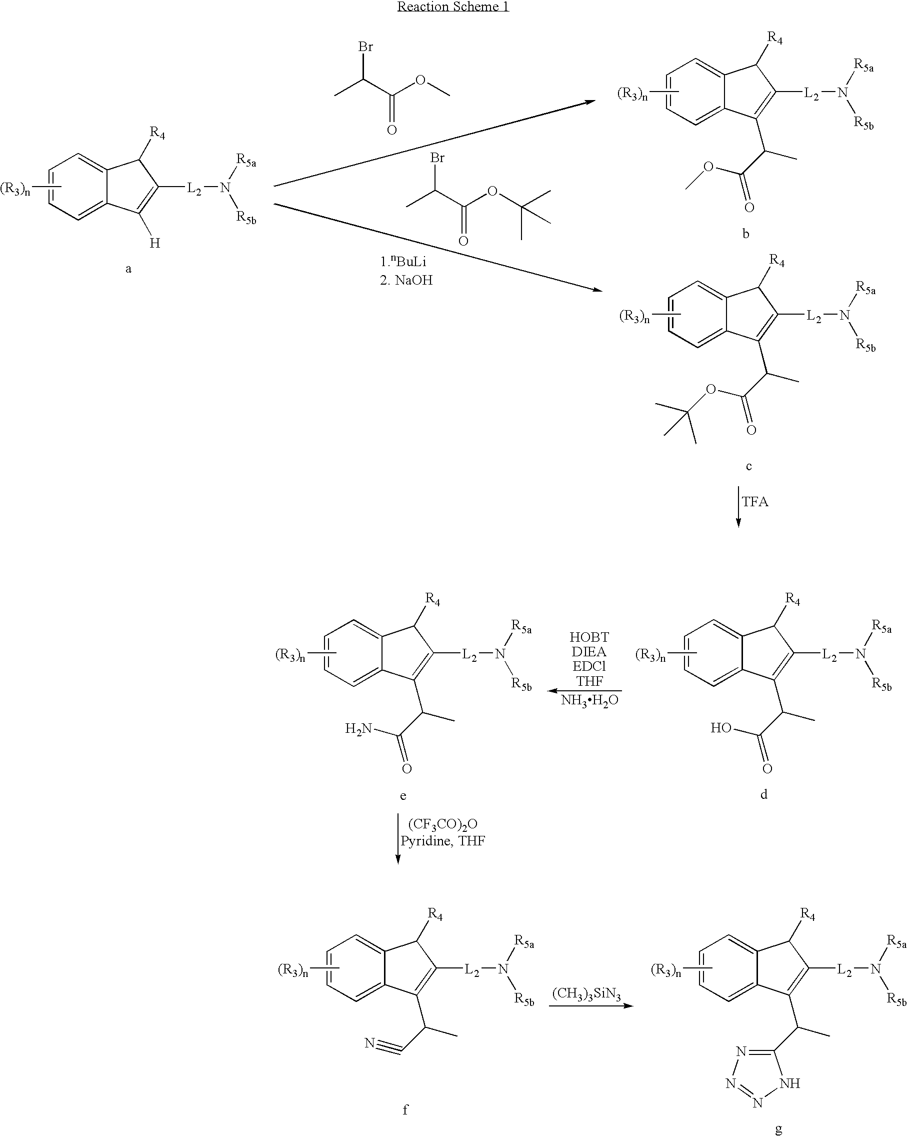

Indene a reacts with 2-bromo-propionic acid methyl ester to form the indene propionic acid methyl ester b. Indene a reacts with 2-bromo-propionic acid t-butyl ester in the presence of n-butyllithium and base to form the protected indene propionic acid ester c. Reaction of compound c with TFA gives rise to the cleavage of the t-butyl group to afford the free acid d. Compound d reacts with HOBt, DIEA, EDCl and NH₃:H₂O in THF to yield the amide e. Reagent e reacts with trifluoroacetic acid anhydride in the presence of pyridine in THF to afford nitrile f which further reacts with trimethylsilylazide to afford tetrazole g.

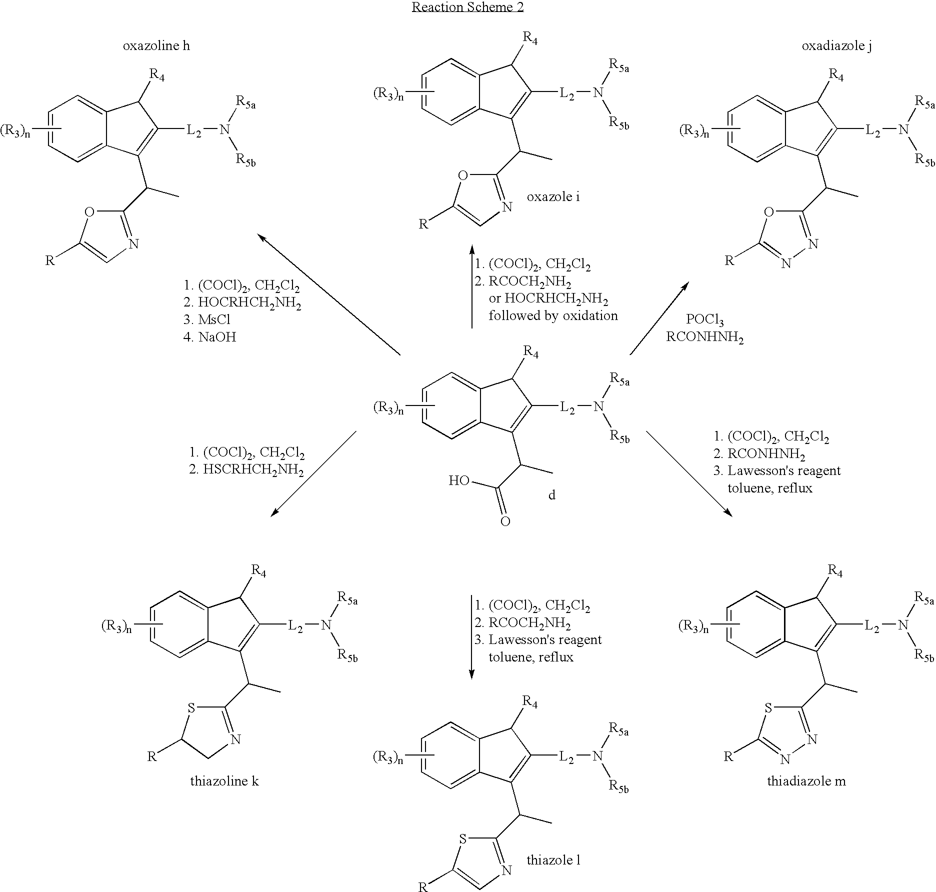

Heterocyclic ring adds to indene propionic acid d (Reaction Scheme 1) via the reactions illustrated above (Reaction Scheme 2.) Reagent d reacts with (COCl)₂ and the amino ethanol in CH₂Cl₂ followed by conversion to the mesylate and treatment with NaOH to yield oxazoline h. Reaction of d with (COCl)₂ and the amino ketone or the amino alcohol followed by oxidation affords the oxazole i. Reaction of d with POCl₃ and the acid hydrazide yields the oxadiazole j. Reaction of d with (COCl)₂ and the amino thiol in CH₂Cl₂ affords thiazoline k. Reaction of d with (COCl)₂ and the amino ketone followed by reflux with Lawesson's reagent in toluene affords thiazole 1. Reaction of d with (COCl)₂ and the acid hydrazide followed by reflux with Lawesson's reagent in toluene affords thiadiazole m.

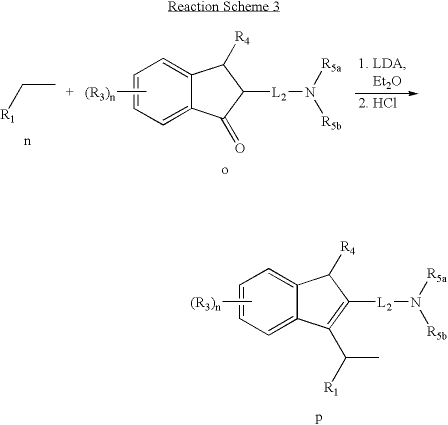

To a solution of LDA is added with stirring ethyl heterocycle reagent n. A solution of indanone o is added with stirring. At the completion of the reaction, water is added to quench. The mixture is extracted with Et₂O, washed with aqueous NaHCO₃ solution, and extracted with HCl aqueous solution. The combined HCl layers are heated. After cooling to room temperature, the mixture is neutralized with cooled NH₄OH and extracted with EtOAc. The organic layer is dried over Na₂SO₄ and concentrated to afford compound p.

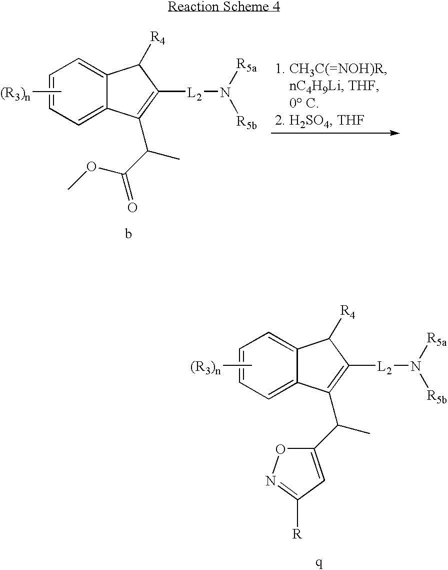

Reagent b (Reaction Scheme 1) reacts with a mixture of the oxime and n-butyllithium in THF at low temperature and then with H₂SO₄ in THF to yield the isoxazole q.

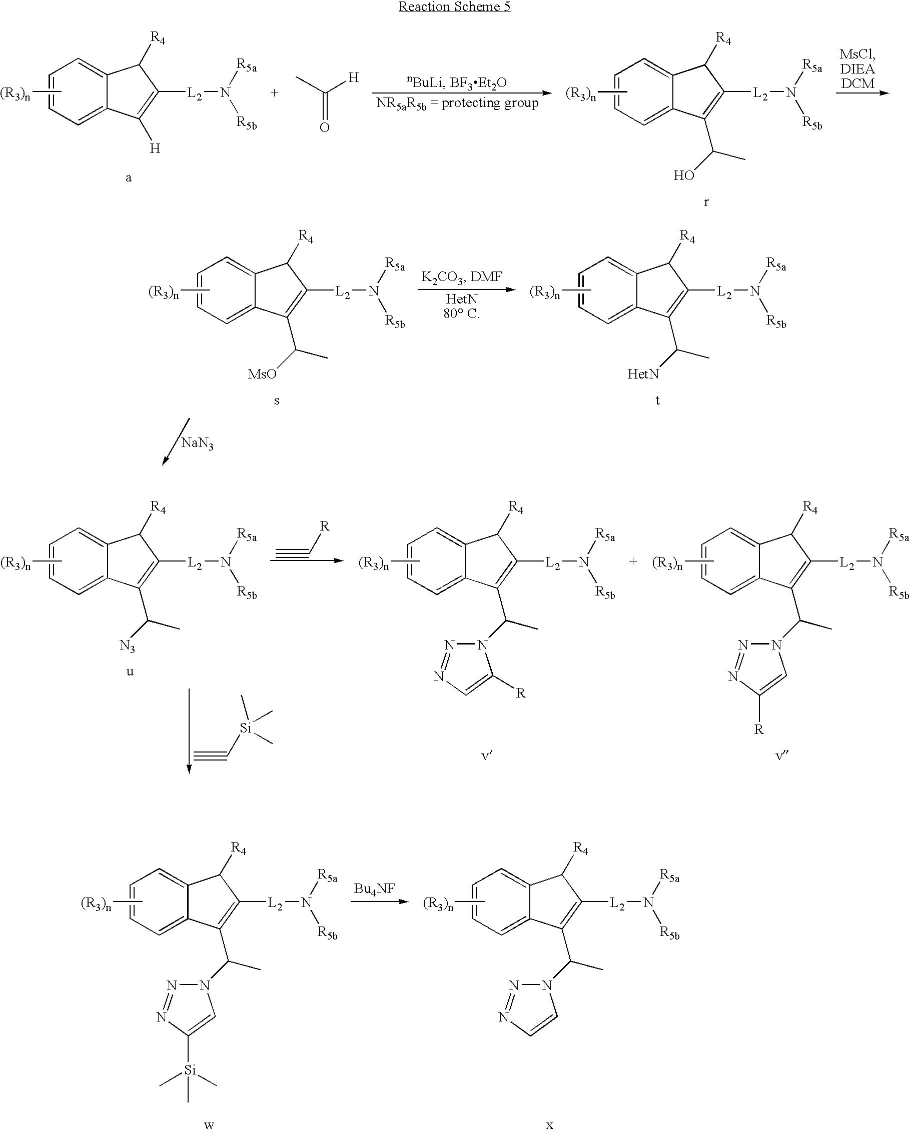

Indene a (Reaction Scheme 1) reacts with acetaldehyde, n-butyllithium, and BF₃ in ET₂O to form alcohol reagent r. Reagent r reacts with methanesulfonyl chloride and DIEA in dichloromethane to afford protected reagent s. Addition of heterocyclic reagent “HetN,” wherein said heterocyclic reagent contains a nitrogen with a dissociable proton in the ring, to reagent s affords compound t. Reaction of azide with reagent s affords reagent u. Addition of substituted acetylene to reagent u affords the substituted triazole compounds v′ and v″. Reaction of reagent u with (trimethylsilyl)acetylene affords reagent w which undergoes trimethysilyl loss resulting from reaction with tetrabutylammonium fluoride to afford triazole compound x.

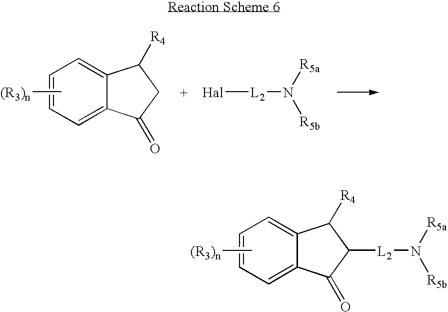

Alkylation of an appropriate 1-indanone with hal-L₂-N(R₅ₐR_{5b}) in the presence of a strong base such as lithium diisopropylamide in a solvent such as THF gives the 2-substituted indanone.

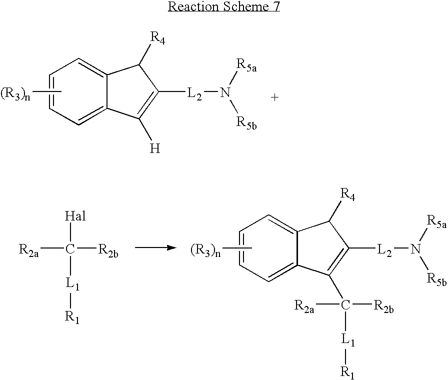

An appropriately substituted indene may be alkylated with a compound such as halogen—C(R₂ₐR_{2b})-L₁-R₁ in the presence of an alkyllithium catalyst in a solvent such as THF.

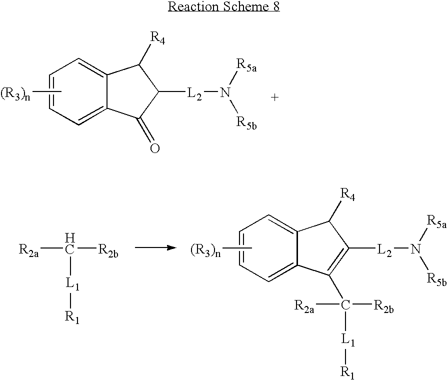

Alkylation of an appropriate 1-indanone with H-L₂-NR₅ₐR_{5b} in the presence of a strong base such as lithium diisopropylamide in a solvent such as THF gives a substituted indene.

For the purposes of administration, the compounds of the present invention may be formulated as pharmaceutical compositions. Pharmaceutical compositions of the present invention comprise a compound of structures (I), (II) or (III) and a pharmaceutically acceptable carrier and/or diluent. The compound is present in the composition in an amount that is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface active agents, binders, and lubricants. One skilled in this art may further formulate the compound in an appropriate manner, and in accordance with accepted practices, such as those disclosed in Remington's Pharmaceutical Sciences, Gennaro, Ed., Mack Publishing Co., Easton, Pa. 1990.

The compounds of this invention may be evaluated for their ability to bind to histamine receptor ligands, which may be determined by techniques known in this field. For example, Example 26 provides a general procedure for calculating the binding to the histamine H₁ receptor by a standard binding assay, while Example 27 provides a general procedure for determining the sedative effects of test compounds employing electroencephalography and electromyography.

In other embodiments, the present invention provides a method for treating a condition related to a histamine receptor. Such methods include administration of a compound of structure (I), (II) or (III) to a warm-blooded animal (including a human) in an amount sufficient to treat the condition. In this context, “treat” includes prophylactic administration. Such methods include systemic administration of compound of this invention, typically in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration and not limitation.

### EXAMPLES

Analytical HPLC-MS Method 1

Platform: Agilent 1100 series: equipped with an auto-sampler, an UV detector (220 nM and 254 nM), a MS detector (APCI);

HPLC column: YMC ODS AQ, S-5, 5μ, 2.0×50 mm cartridge;

HPLC gradient: 1.0 mL/minute, from 10% acetonitrile in water to 90% acetonitrile in water in 2.5 minutes, maintaining 90% for 1 minute. Both acetonitrile and water have 0.025% TFA.

Analytical HPLC-MS Method 2

Platform: Agilent 1100 series: equipped with an auto-sampler, an UV detector (220 nM and 254 nM), a MS detector (APCI);

HPLC column: Phenomenex Synergi-Max RP, 2.0×50 mm column;

HPLC gradient: 1.0 mL/minute, from 5% acetonitrile in water to 95% acetonitrile in water in 13.5 minutes, maintaining 95% for 2 minute. Both acetonitrile and water have 0.025% TFA.

Analytical HPLC-MS Method 3

Platform: Agilent 1100 series: equipped with an auto-sampler, an UV detector (220 nM and 254 nM), a MS detector (electrospray);

HPLC column: XTerra MS, C₁₈, 5μ, 3.0×250 mm column;

HPLC gradient: 1.0 mL/minute, from 10% acetonitrile in water to 90% acetonitrile in water in 46 minutes, jump to 99% acetonitrile and maintain 99% acetonitrile for 8.04 minutes. Both acetonitrile and water have 0.025% TFA.

Analytical HPLC-MS Method 4

Platform: Agilent 1100 series: equipped with an auto-sampler, an UV detector (220 nM and 254 nM), a MS detector (APCI) and Berger FCM 1200 CO₂ pump module;

HPLC column: Berger Pyridine, PYR 60A, 6μ, 4.6×150 mm column;

HPLC gradient: 4.0 mL/minute, 120 bar; from 10% methanol in supercritical CO₂ to 60% methanol in supercritical CO₂ in 1.67 minutes, maintaining 60% for 1 minute. Methanol has 1.5% water. Backpressure regulated at 140 bar.

Preparative HPLC-MS

Platform: Shimadzu HPLC equipped with a Gilson 215 auto-sampler/fraction collector, UV detector and a PE Sciex API150EX mass detector;

HPLC column: BHK ODS-O/B, 5μ, 30×75 mm

HPLC gradient: 35 mL/minute, 10% acetonitrile in water to 100% acetonitrile in 7 minutes, maintaining 100% acetonitrile for 3 minutes, with 0.025% TFA.
t_{R}=retention time (in minutes)
Chiral HPLC

Platform: Dionex P680A and P680P pumps, Dionex PAD 100 photodiode array detector, Jasco CD 2095 plus chiral detector, Gilson 215 liquid handler p HPLC Columns: Chiral Technologies, Chiralpak AD-H (chiral profiles 1-8), Chiralcel OD-H (chiral profile 9). Analytical Columns are 0.46×25 cm, 5 μm; preparative columns are 2×25 cm, 5 μm.

Isocratic elutant: Flow Rate: 0.3 to 1.0 mL/min for analytical and 8 to 15 mL/min for preparative.

Elutant profile 1: Hexane/isopropyl alcohol 99/1 with 0.1% isopropylamine

Elutant profile 2: Hexane/isopropyl alcohol 97/3 with 0.1% isopropylamine

Elutant profile 3: Hexane/ethyl alcohol 97/3 with 0.1% diethylamine

Elutant profile 4: Hexane/isopropyl alcohol 95/5 with 0.1% diethylamine

Elutant profile 5: Hexane/isopropyl alcohol 85/15 with 0.1% diethylamine

Elutant profile 6: Hexane/ethyl alcohol 95/5 with 0.1% diethylamine

Elutant profile 7: Hexane/ethyl alcohol 9/1 with 0.1% diethylamine

Elutant profile 8: Hexane/isopropyl alcohol 9/1 with 0.1% diethylamine

Elutant profile 9: Methanol with 0.1% diethylamine.

Abbreviations

LDA: Lithium diisopropylamide

THF: Tetrahydrofuran

HPLC: High performance liquid chromatography

TFA: Trifluoroacetic acid

CHO: Chinese hamster ovary

EDCI: 1-(3-dimethylaminopropyl)-3-ethylcarbodiimide hydrochloride

HOBT: 1-Hydroxybenzotriazole

DIEA: Diisopropylethylamine

MsCl: Methanesulfonyl Chloride

### Example 1

### SYNTHESIS OF REAGENT N,N-DIMETHYL-2-(1-OXO-INDAN-2-YL)-ACETAMIDE

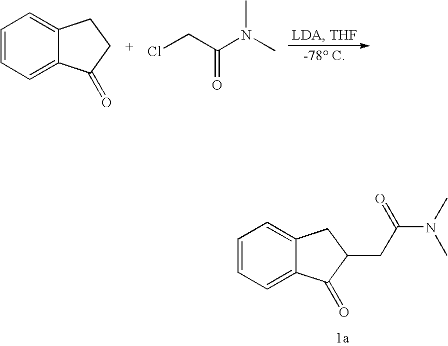

To a solution of 1-indanone (26.4 g, 200 mmol) in THF (400 mL) at −78° C., LDA (100 mL, 2 M in THF/toluene, 200 mmol) was added. After stirring at −78° C. for an hour, 2-chloro-N,N-dimethylacetamide (24.4 g, 200 mmol) was added. The resultant mixture was stirred at −78° C. for 3 hours and then warmed to room temperature overnight. NH₄Cl (saturated, 200 mL) was added to quench the reaction. The mixture was extracted with EtOAc (2×250 mL). The combined organic solution was washed with water (200 mL), dried over Na₂SO₄, concentrated and purified by flash chromatography (hexane:EtOAc, 3:2) to give N, N-dimethyl-2-(1-oxo-indan-2-yl)-acetamide 1a as a pale yellow oil,41% yield. (MH⁺=218.3)

The following compounds were also made according to this procedure:
2-(5-Chloro-1-oxo-indan-2-yl)-N,N-dimethyl-acetamide 1b, 38% yield. (MH⁺=251.9)
N,N-Dimethyl-2-(5-methyl-1-oxo-indan-2-yl)-acetamide 1c, 22% yield. (MH⁺=231.9)
2-(5-Methoxy-1-oxo-indan-2-yl)-N,N-dimethyl-acetamide 1d, 60% yield. (MH⁺=247.9)
2-(5-Fluoro-1-oxo-indan-2-yl)-N,N-dimethyl-acetamide 1e, 42% yield. (MH⁺=235.9)

### Example 2

### SYNTHESIS OF REAGENT 2-(2-DIMETHYLAMINO-ETHYL)-INDAN-1-ONE

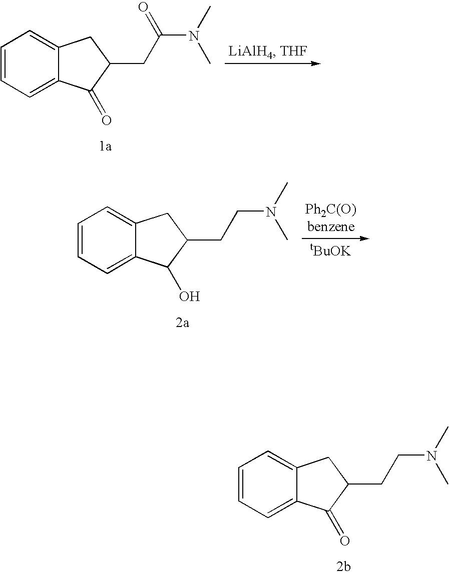

To a solution of LiAlH₄ (7.8 g, 200 mmol) in THF (300 mL) at 0° C., N,N-dimethyl-2-(1-oxo-indan-2-yl)-acetamide 1a (10 g, 46 mmol) in THF (40 mL) was added slowly. The reaction was allowed to stir at room temperature for half an hour and then at reflux for 6 hours. The reaction was cooled to room temperature and carefully quenched by the sequential addition of H₂O (7.6 mL), 15% NaOH (7.6 mL) and H₂O (22.8 mL). The mixture was stirred for a half hour, and the solid was filtered off using a celite pad and washed with excess THF. The combined filtrates were dried over Na₂SO₄ and concentrated to provide 2-(2-dimethylamino-ethyl)-indan-1-ol 2a as a yellow oil. A mixture of 2-(2-dimethylamino-ethyl)-indan-1-ol 2a, ^{t}BuOK (11.2 g, 100 mmol) and benzophenone (36.4 g,200 mmol) in anhydrous benzene (200 mL) was refluxed for 18 hours under N₂. The cooled mixture was poured into ice and extracted with 10% HCl until the HCl solution was colorless. The combined acid extracts were washed with Et₂O (300 mL) and added dropwise with stirring into NH₄OH and ice. This basic solution was extracted thrice with Et₂O (300 mL). The combined organic layers were washed with brine, dried over Na₂SO₄, concentrated and purified by flash chromatography (CHCl₃:MeOH:NH₄OH, 95:5:0.2) to give 2-(2-dimethylamino-ethyl)-indan-1-one 2b as a pale yellow oil in 79% yield. (MH⁺=204.2).

The following compounds were also made according to this procedure:
2-(2-Dimethylamino-ethyl)-5-fluoro-indan-1-one 2c, 42% yield. (MH⁺=221.9)
2-(2-Dimethylamino-ethyl)-5-chloro-indan-1-one 2d, 81% yield. (MH⁺=237.9)
2-(2-Dimethylamino-ethyl)-5-methyl-indan-1-one 2e, 62% yield. (MH⁺=217.9)
2-(2-Dimethylamino-ethyl)-5-methoxy-indan-1-one 2f, 54% yield. (MH⁺=233.9)

### Example 3

### SYNTHESIS OF REAGENT [2-(1H-INDEN-2-YL)-ENTHYL]-DIMETHYL-AMINE

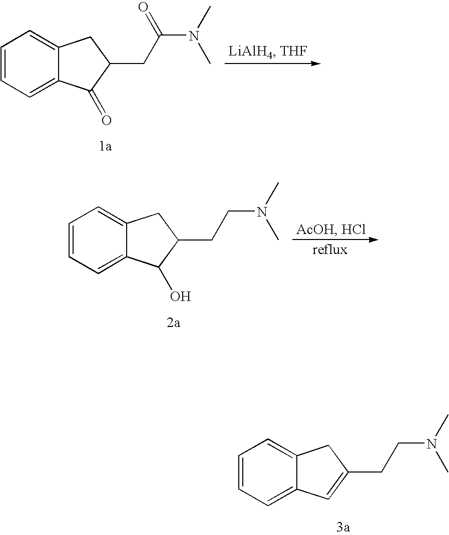

To a solution of LiAlH₄ (7.8 g, 200 mmol) in THF (300 mL) at 0° C., N,N-dimethyl-2-(1-oxo-indan-2-yl)-acetamide 1a (10 g, 46 mmol) in THF (40 mL) was added slowly. The reaction was allowed to stir at room temperature for half an hour and then at reflux for 6 hours. The reaction was cooled to room temperature and carefully quenched by the sequential addition of H₂O (7.6 mL), 15% NaOH (7.6 mL) and H₂O (22.8 mL). The mixture was stirred for a half hour, and the solid was filtered off using a celite pad and washed with excess THF. The combined filtrates were dried over Na₂SO₄ and concentrated to provide 2-(2-dimethylamino-ethyl)-indan-1-ol 2a as a yellow oil. 2-(2-Dimethylamino-ethyl)-indan-1-ol 2a was dissolved in acetic acid (120 mL) and concentrated HCl (40 mL), and the resultant mixture was refluxed for 2 hours. Most of the solvent was evaporated in vacuo. The residue was diluted with water (200. mL) and washed with ether (50 mL). The aqueous solution was basified with NH₄OH, extracted twice with ether (250 mL), dried over Na₂SO₄, concentrated and purified by flash chromatography (CHCl₃:MeOH:NH₄OH, 98:2:0.2) to give a pale yellow oil, [2-(1H-inden-2-yl)-ethyl]-dimethyl-amine 3a, in 83% yield. (MH⁺=188.0)

In a similar manner [2-(6-methoxy-1H-inden-2-yl)-ethyl]-dimethyl-amine 3b was prepared in 15% yield. MH⁺=217.9

### Example 4

### DIMETHYL-{2-[3-(1-PYRAZIN-2-YL-ETHYL)-1H-INDEN-2-YL]-ETHYL}-AMINE

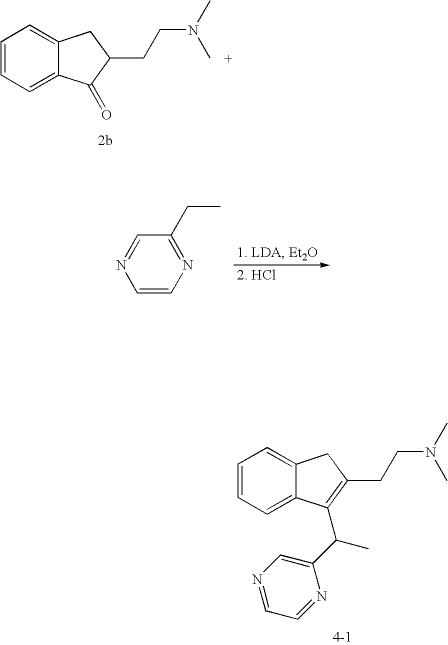

To a solution of LDA (1 mL, 2 N solution in THF, 2 mmol) in Et₂O (5 mL) at 0° C., 2-ethylpyrazine (238 mg, 2.2 mmol) in Et₂O (1 mL) was added slowly, and the reaction mixture was stirred at 0° C. for 30 minutes. A solution of 2-(2-dimethylamino-ethyl)-indan-1-one 2b (203 mg, 1 mmol) in Et₂O (1 mL) was added. The resultant mixture was stirred at 0° C. for two hours. A small piece of chip ice was added to quench the reaction. The mixture was extracted with Et₂O, washed with NaHCO₃ aqueous solution, then extracted with 20% HCl (2×5 mL). The combined HCl layers were heated to 100° C. for 1 hour. After cooling to room temperature, the mixture was neutralized with cooled NH₄OH and extracted with EtOAc. The organic layer was dried over Na₂SO₄ and concentrated to give dimethyl-{2-[3-(1-pyrazin-2-yl-ethyl)-1H-inden-2-yl]-ethyl}amine 4-1 as a red oil which was purified by HPLC yielding the TFA salt. Chiral HPLC separation on 4-1 using a Chiraltech Chiralpak AD-H column and hexane:isopropyl alcohol 99:1 (with 0.1% isopropylamine) as elutant yielded enantiomers 4-5 and 4-6.

The following compounds were made according to this procedure:

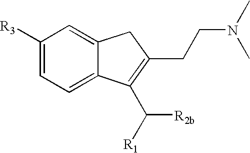

| No. | R₁ | R_{2b} | R₃ | MW | MH⁺ | t_{R} (method) |
|---|---|---|---|---|---|---|
| 4-1 | Pyrazin-2-yl | CH₃ | H | 293.41 | 294.2 | 3.700 (2) |
| 4-2 | Pyrimidin-4-yl | CH₃ | H | 293.41 | 294.0 | 3.997 (2) |
| 4-3 | Pyrazin-2-yl | H | H | 279.38 | 280.0 | 3.441 (2) |
| 4-4 | 3-methoxy-pyrazin-2-yl | CH₃ | H | 323.44 | 324.1 | 4.602 (2) |
| 4-5 | Pyrazin-2-yl | (S)-CH₃ | H | 293.41 | 294.1 | 4.091 (2) |
| | | profile 1 | | | | |
| 4-6 | Pyrazin-2-yl | (R)-CH₃ | H | 293.41 | 294.1 | 4.057 (2) |
| | | profile 1 | | | | |
| 4-7 | 3-methoxy-pyrazin-2-yl | (R-)CH₃ | H | 323.44 | 323.9 | 4.443 (2) |
| | | profile 2 | | | | |
| 4-8 | 3-methoxy-pyrazin-2-yl | (S)-CH₃ | H | 323.44 | 323.9 | 4.425 (2) |
| | | profile 2 | | | | |
| 4-9 | 3-methyl-pyrazin-2-yl | H | H | 293.41 | 294.0 | 3.530 (2) |
| 4-10 | pyridazin-3-yl | H | H | 279.38 | 280.0 | 3.023 (2) |
| 4-11 | 3-methoxy-pyrazin-2-yl | H | H | 309.41 | 310.0 | 4.279 (2) |
| 4-12 | pyrazin-2-yl | CH₃, CH₃ | H | 307.44 | 308.1 | 3.883 (2) |
| 4-13 | 5-methyl-pyrazin-2-yl | H | H | 293.41 | 294.0 | 3.823 (2) |
| 4-14 | 6-methyl-pyrazin-2-yl | H | H | 293.41 | 294.0 | 3.801 (2) |
| 4-15 | pyrimidin-2-yl | H | H | 279.38 | 280.0 | 3.323 (2) |
| 4-16 | 3-ethoxy-pyrazin-2-yl | H | H | 323.44 | 323.9 | 4.861 (2) |
| 4-17 | 6-propoxy-pyrazin-2-yl | H | H | 337.46 | 337.9 | 5.300 (2) |
| 4-18 | pyrazin-2-yl | H | Cl | 313.83 | 314.0 | 4.223 (2) |
| 4-19 | 3-methoxy-pyrazin-2-yl | H | Cl | 343.86 | 344.1 | 18.71 (3) |
| 4-20 | 3-methoxy-pyrazin-2-yl | CH₃ | Cl | 357.88 | 358.1 | 19.66 (3) |
| 4-21 | pyridazin-3-yl | H | Cl | 313.83 | 313.8 | 4.027 (2) |
| 4-22 | 3-methoxy-pyrazin-2-yl | (S)-CH₃ | Cl | 357.88 | 357.8 | 5.195 (2) |
| | | profile 3 | | | | |
| 4-23 | 3-methoxy-pyrazin-2-yl | (R)-CH₃ | Cl | 357.88 | 357.8 | 5.177 (2) |
| | | profile 3 | | | | |
| 4-24 | pyrazin-2-yl | H | CH₃ | 293.41 | 293.9 | 4.008 (2) |
| 4-25 | pyridazin-3-yl | H | CH₃ | 293.41 | 294.1 | 3.862 (2) |
| 4-26 | pyrazin-2-yl | CH₃ | CH₃ | 307.44 | 307.9 | 4.366 (2) |
| 4-27 | 3-methoxy-pyrazin-2-yl | CH₃ | CH₃ | 337.46 | 337.9 | 5.113 (2) |
| 4-28 | pyrazin-2-yl | (R)-CH₃ | CH₃ | 307.44 | 307.9 | 4.337 (2) |
| | | profile 4 | | | | |
| 4-29 | pyrazin-2-yl | (S)-CH₃ | CH₃ | 307.44 | 307.9 | 4.336 (2) |
| | | profile 4 | | | | |
| 4-30 | 3-methoxy-pyrazin-2-yl | (R)-CH₃ | CH₃ | 337.46 | 337.9 | 5.087 (2) |
| | | profile 5 | | | | |
| 4-31 | 3-methoxy-pyrazin-2-yl | (S)-CH₃ | CH₃ | 337.46 | 337.9 | 5.100 (2) |
| | | profile 5 | | | | |
| 4-32 | pyrazin-2-yl | H | F | 297.38 | 297.9 | 3.706 (2) |
| 4-33 | pyridazin-3-yl | H | F | 297.38 | 297.9 | 3.308 (2) |
| 4-34 | 3-methoxy-pyrazin-2-yl | H | F | 327.40 | 327.9 | 4.558 (2) |
| 4-35 | 3-methoxy-pyrazin-2-yl | Me | F | 341.43 | 341.9 | 4.833 (2) |
| 4-36 | 3-methoxy-pyrazin-2-yl | (R)-CH₃ | F | 341.43 | 341.9 | 4.919 (2) |
| | | profile 6 | | | | |
| 4-37 | 3-methoxy-pyrazin-2-yl | (S)-CH₃ | F | 341.43 | 341.8 | 4.878 (2) |
| | | profile 6 | | | | |
| 4-38 | 3-ethoxy-pyrazin-2-yl | Me | F | 355.45 | 356.1 | 5.212 (2) |
| 4-39 | 3-methoxy-pyrazin-2-yl | Et | F | 355.45 | 356.1 | 5.294 (2) |
| 4-40 | pyrazin-2-yl | H | OCH₃ | 309.41 | 310.1 | 3.415 (2) |
| 4-41 | pyrazin-2-yl | Me | OCH₃ | 323.44 | 323.9 | 3.947 (2) |
| 4-42 | pyridazin-3-yl | H | OCH₃ | 309.41 | 309.9 | 3.253 (2) |
| 4-43 | 3-methoxy-pyrazin-2-yl | Me | OCH₃ | 353.46 | 353.9 | 4.565 (2) |
| 4-44 | 3-methoxy-pyrazin-2-yl | (S)-CH₃ | OCH₃ | 353.46 | 354.1 | 4.563 (2) |
| | | profile 7 | | | | |
| 4-45 | 3-methoxy-pyrazin-2-yl | (R)-CH₃ | OCH₃ | 353.46 | 354.1 | 4.533 (2) |
| | | profile 7 | | | | |
| 4-46 | pyrazin-2-yl | (R)-CH₃ | OCH₃ | 323.44 | 323.9 | 3.710 (2) |
| | | profile 7 | | | | |
| 4-47 | pyrazin-2-yl | (S)-CH₃ | OCH₃ | 323.44 | 323.9 | 3.774 (2) |
| | | profile 7 | | | | |
| 4-48 | 3-hydroxy-pyrazin-2-yl | CH₃ | H | 309.4 | 310.1 | 3.657 (2) |
| 4-49 | 3-hydroxy-pyrazin-2-yl | (R)-CH₃ | F | 327.4 | 328.0 | 3.941 (2) |
| 4-50 | 3-ethoxy-pyrazin-2-yl | (R)-CH₃ | F | 355.45 | 356.1 | 5.261 (2) |
| | | profile 4 | | | | |
| 4-51 | 3-ethoxy-pyrazin-2-yl | (S)-CH₃ | F | 355.45 | 356.1 | 5.286 (2) |
| | | profile 4 | | | | |

### Example 5

### DIMETHYL-{2-[3-(1-THIAZOL-2-YL-ETHYL)-1H-INDEN-2-YL]-ETHYL}-AMINE

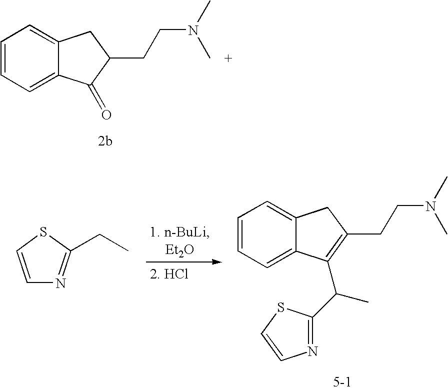

To a solution of 2-ethylthiazole (250 mg, 2.2 mmol) in Et₂O (4 mL) at −78° C., n-BuLi (1.3 mL, 1.6 N solution in n-hexane, 2 mmol) was added, and the reaction mixture was stirred at −78° C. for 2 hours. A solution of 2-(2-dimethylamino-ethyl)-indan-1-one 2b (203 mg, 1 mmol) in Et₂O (1 mL) was added. The resultant mixture was stirred at −78° C. for 5 hours. A small piece of ice chip was added to quench the reaction at −78° C. The mixture was extracted with Et₂O, washed with NaHCO₃ aqueous solution, then extracted with 20% HCl (2×5 mL). The combined HCl layers were heated to 100° C. for 1 hour. After cooling to room temperature, the mixture was neutralized with cooled NH₄OH and extracted with EtOAc. The organic layers were dried over Na₂SO₄ and concentrated to give a red oil, which was purified by HPLC to give dimethyl-{2-[3-(1-thiazol-2-yl-ethyl)-1H-inden-2-yl]-ethyl}-amine 5-1 as a TFA salt (157 mg, 38% yield). (MH⁺=299.1). Chiral HPLC gave enantiomers 5-2 and 5-3.

2-Ethyl-5-trimethylsilanyl-thiazole may be used in place of 2-ethylthiazole as shown in the alternative procedure below. To a solution of 2-ethylthiazole (2.26 g, 20 mmol) in THF (50 mL) at −50° C. under N₂, nBuLi (12.5 mL, 1 M solution in hexanes, 20 mmol) was added. The solution was stirred at −50° C. for 30 min and chlorotrimethylsilane (2.61 g, 24 mmol, 1.2 eq.) was added dropwise. After removal of the cooling bath, the mixture was allowed to slowly reach room temperature overnight. The reaction was quenched with sat. NaHCO₃ and extracted with EtOAc, dried over Na₂SO₄, concentrated and distilled (45-48° C./10 mmHg) to give 1.3 g of 2-ethyl-5-trimethylsilanyl-thiazole (33% yield).

To a cooled solution (−78° C.) of 2-ethyl-5-trimethylsilanyl-thiazole (0.243 g, 1.32 mmol) in THF (2 mL), n-BuLi (0.82 mL, 1.6 M) was slowly added. The solution was stirred for 30 min at −78° C. A solution of 2-(2-dimethylamino-ethyl)-5-methyl-indan-1-one 2e (0.143 g, 0.66 mmol) in THF (2 mL) was added slowly and stirring was continued for 16 hrs while the temperature slowly increased to room temperature. The solvent was removed in vacuo and dichloromethane was added. The organic layer was washed with water, dried (MgSO₄), filtered and concentrated in vacuo. The residue was treated with 20% HCl (3 mL) for 5 hours at room temperature. Neutralization with NH₄OH was followed by extraction with dichloromethane. The separated organic layer was washed with water and concentrated in vacuo. Purification by preparative HPLC afforded dimethyl-(2-{6-methyl-3-[1-(5-trimethylsilanyl-thiazol-2-yl)-ethyl]-1H-inden-2-yl}-ethyl)-amine as a TFA salt. MW=370.6, MH⁺=371,; t_{R}=5.398 (method 2). The salt was neutralized by ammonia and extracted with dichloromethane. The organic layer was dried over MgSO₄, filtered and concentrated to give 0.12 g of free base (47% yield) which was dissolved in acetonitrile and a 48-51% aqueous solution of HF (0.12 g, 3.1 mmol) was added. The reaction mixture was stirred overnight at room temperature, neutralized with ammonia and extracted with dichloromethane. The combined organic layers were dried (MgSO₄), filtered and concentrated in vacuo to give 5-1. The enantiomers were separated using chiral HPLC.

The following compounds were made according to this procedure:

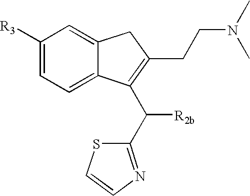

| No. | R₃ | R_{2b} | MW | MH⁺ | t_{R} (method 2) |
|---|---|---|---|---|---|
| 5-1 | —H | CH₃ | 298.45 | 299 | 4.238 |
| 5-2 | —H | (R)-CH₃ | 298.45 | 299 | 4.292 |
| | | profile 6 | | | |
| 5-3 | —H | (S)-CH₃ | 298.45 | 299 | 4.299 |
| | | profile 6 | | | |
| 5-4 | —CH₃ | (R)-CH₃ | 312.48 | 313 | 4.654 |
| | | profile 7 | | | |
| 5-5 | —CH₃ | (S)-CH₃ | 312.48 | 313 | 4.619 |
| | | profile 7 | | | |
| 5-6 | —OCH₃ | —CH₃ | 328.48 | 328.8 | 4.234 |
| 5-7 | —OCH₃ | (R)-CH₃ | 328.48 | 329 | 4.310 |
| | | profile 6 | | | |
| 5-8 | —OCH₃ | (S)-CH₃ | 328.48 | 329 | 4.325 |
| | | profile 6 | | | |
| 5-9 | —F | CH₃ | 316.44 | 317 | 4.503 |
| 5-10 | —F | (R)-CH₃ | 316.44 | 317 | 4.524 |
| | | profile 7 | | | |
| 5-11 | —F | (S)-CH₃ | 316.44 | 317 | 4.495 |
| | | profile 7 | | | |

### Example 6

### {2-[3-(2-ETHOXY-1-METHYL-ETHYL)-1H-INDEN-2-YL]-ETHYL}-DIMETHYL-AMINE

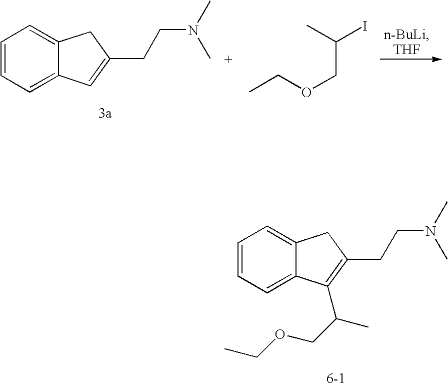

To a solution of [2-(1H-inden-2-yl)-ethyl]-dimethyl-amine 3a (28 mg, 0.15 mmol) in THF (1.5 mL) at −78° C., n-butyllithium (0.1 mL, 1.6 M solution in n-hexane, 0.16 mmol) was added. After stirring at −78° C. for 1 hour, 2-iodopropyl ethyl ether (42 mg, 0.2 mmol) was added. The resultant mixture warmed to room temperature overnight. Water (5 mL) and Et₂O (5 mL) were added. The organic layer was isolated, dried over Na₂SO₄ and concentrated to give an oil, which was purified by HPLC to give {2-[3-(2-ethoxy-1-methyl-ethyl)-1H-inden-2-yl]-ethyl}-dimethyl-amine 6-1 as the TFA salt, 6% yield. (MH⁺=274.3)

The following compounds were made according to this procedure:

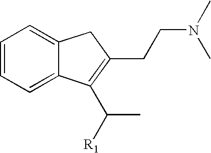

| No. | R₁ | MW | MH⁺ | t_{R} (method 2) |
|---|---|---|---|---|
| 6-1 | —CH₂OCH₂CH₃ | 273.42 | 274.3 | 5.012 |
| 6-2 | —CH₂OCH₃ | 259.39 | 260.1 | 4.225 |

### Example 7

### 2-[2-(2-DIMETHYLAMINO-ETHYL)-3H-INDEN-1-YL]-PROPIONIC ACID TERT-BUTYL ESTER

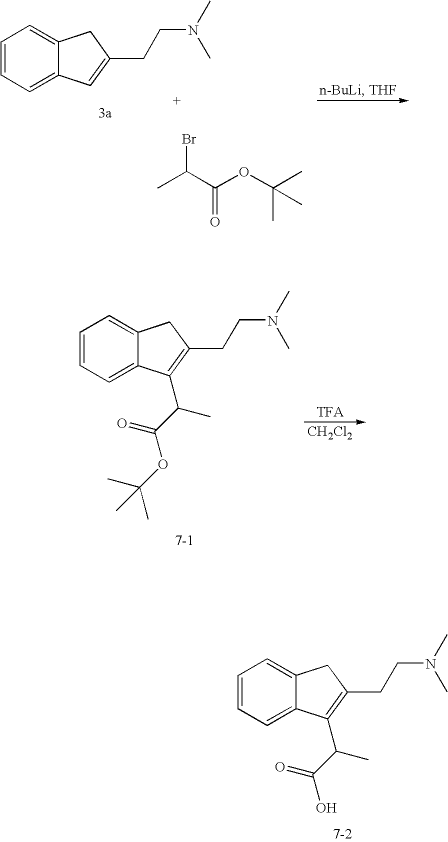

To a solution of [2-(1H-inden-2-yl)-ethyl]-dimethyl-amine 3a (28 mg, 0.15 mmol) in THF (1.5 mL) at −78° C., n-butyllithium (100 μL, 0.16mmol, 1.6M in hexanes) was added. After stirring at −78° C. for 1 hour, 2-bromo-propionic acid tert-butyl ester (0.2 mmol) was added. The resultant mixture was warmed to room temperature overnight. Saturated NaHCO₃ (5 mL) and Et₂O (5 mL) were added. The organic layer was isolated and dried over Na₂SO₄ and concentrated to give an oil, which was purified by HPLC to give 2-[2-(2-dimethylamino-ethyl)-3H-inden-1-yl]-propionic acid tert-butyl ester as the TFA salt: compound 7-1. (MH⁺=316.2). To a solution of compound 7-1 (9.42 mmol) in dichloromethane (10 mL) at 0° C., TFA (5 mL) was added. After stirring at room temperature overnight, the resultant mixture was concentrated in vacuo. Ethyl acetate was added to the residue with sonication. 2-[2-(2-Dimethylamino-ethyl)-3H-inden-1-yl]-propionic acid 7-2 was obtained as a white precipitate TFA salt: 65% yield. (MH⁺=260.0)

The following compounds were made according to this procedure:

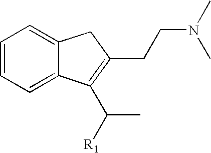

| No. | R₁ | MW | MH⁺ | t_{R} (method 2) |
|---|---|---|---|---|
| 7-1 | —C(O)OC(CH₃)₃ | 315.45 | 316.2 | 5.347 (2) |
| 7-2 | —C(O)OH | 259.35 | 260.0 | 3.369 (2) |
| 7-3 | —C(O)OCH₃ | 273.37 | 274.0 | 1.680 (1) |

### Example 8

### DIMETHYL-(2-{3-[1-(5-METHYL-[1,3,4]OXADIAZOL-2-YL)-ETHYL]-1H-INDEN-2-YL}-ETHYL)-AMINE

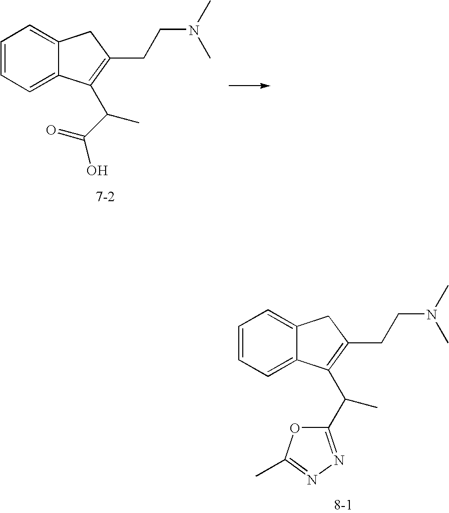

To a solution of compound 7-2 (50 mg, 0.193 mmol) in POCl₃ (5.36 mmol, 0.5 mL) was added N-acetyl hydrazide (16 mg, 0.21 mmol.) The reaction mixture was refluxed under nitrogen atmosphere for 2h, cooled, poured over ice and rendered basic to pH 7-8 using concentrated NH₄OH. To the ice-cold aqueous layer was added 2 mL of brine, and the mixture was extracted with methylene chloride (3×1 mL). The organic layers were combined, dried over MgSO₄, and filtered, and the solvent was removed in vacuo to give a dark oil which was purified by flash column chromatography (NH₄OH:MeOH:CH₂Cl₂, 1:5:94) to give dimethyl-(2-{3-[1-(5-methyl-[1,3,4]oxadiazol-2-yl)-ethyl]-1H-inden-2-yl}-ethyl)-amine 8-1, 31% yield. (MH⁺=298.2)

The following compounds were made according to this procedure:

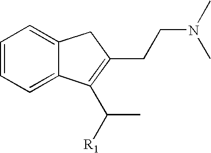

| No. | R₁ | MW | MH⁺ | t_{R} (method 2) |
|---|---|---|---|---|
| 8-1 | 5-methyl-[1,3,4]oxadiazol-2-yl | 297.40 | 298.2 | 3.500 |
| 8-2 | 5-phenyl-[1,3,4]oxadiazol-2-yl | 359.47 | 360.2 | 4.920 |

### Example 9

### DIMETHYL-(2-{3-[1-(5-PHENYL-OXAZOL-2-YL)-ETHYL]-1H-INDEN-2-yl}-ETHYL)-AMINE

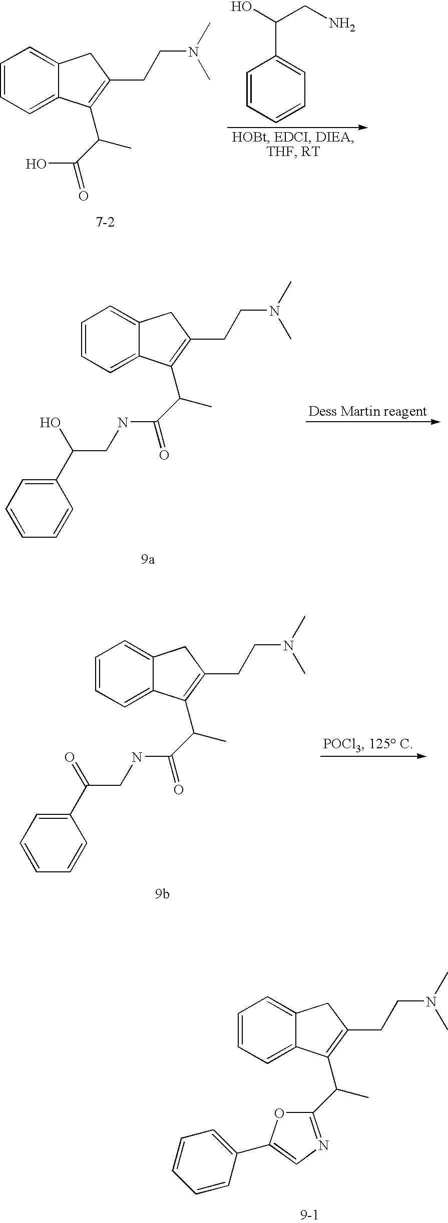

To a solution of 7-2 (99 mg, 0.38 mmol) in THF (1 mL) was added HOBT (78 mg, 0.58 mmol), EDCI (III mg, 0.58 mmol), DIEA (137 μL, 0.77 mmol) and 2-amino-1-phenylethanol (52 mg, 0.38 mmol). The reaction mixture was stirred at room temperature overnight. The reaction mixture was concentrated in vacuo and dissolved in methylene chloride (2 mL). The methylene chloride solution was quenched with brine, dried over MgSO₄, filtered, and the solvent was removed in vacuo to give 2-[2-(2-dimethylamino-ethyl)-3H-inden-1-yl]-N-(2-hydroxy-2-phenyl-ethyl)-propionamide 9a as a yellow oil. The yellow oil was dissolved in methylene chloride (1 mL), and Dess Martin reagent (162 mg,0.38 mmol) was added. The mixture was stirred overnight and quenched with 1N NaOH aqueous solution and brine. The organic solution was concentrated to dryness and purified by flash column chromatography (NH₄OH:MeOH:CH₂Cl₂, 1:5:94) to give compound 9b, 70% yield. (MH⁺=377.2). A solution of compound 9b (68 mg, 0.18 mmol) in POCl₃ (1 mL) was heated at 125° C. under nitrogen for 2 h. The reaction mixture was cooled, poured over ice, and rendered basic to pH 7-8 using concentrated NH₄OH. To the ice-cold aqueous layer was added brine (2 mL) and the mixture was extracted with methylene chloride (3×1 mL). The organic layers were combined, dried over MgSO₄ and filtered, and the solvent was removed in vacuo to give a dark oil which was purified by prep HPLC to give dimethyl-(2-{3-[1-(5-phenyl-oxazol-2-yl)-ethyl]-1H-inden-2-yl}-ethyl)-amine 9-1 as the TFA salt, 13% yield. (MH⁺=359.2)

### Example 10

### DIMETHYL-(2-{3-[1-(5-PHENYL-THIAZOL-2-yl]-ETHYL]-1H-INDEN-2-yl}-ETHYL)-AMINE

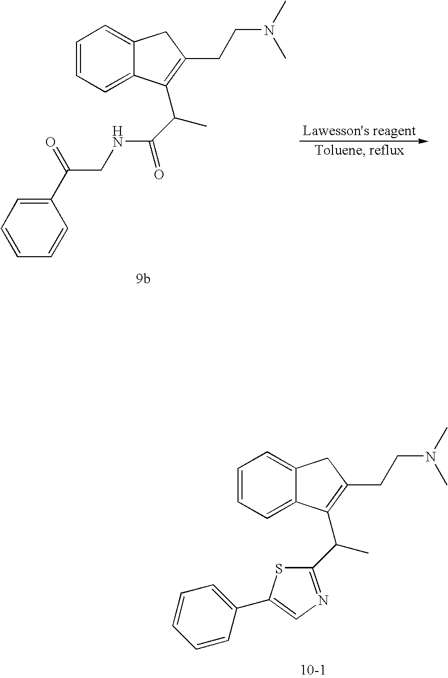

A solution of compound 9b (27 mg, 0.07 mmol) and Lawesson's reagent (113 mg, 0.28 mmol) in toluene (0.5 mL) was refluxed for 4 hours under nitrogen. The reaction mixture was cooled and solvent removed in vacuo to give a yellow residue which was purified by preparative HPLC to give dimethyl-(2-{3-[1-(5-phenyl-thiazol-2-yl)-ethyl]-1H-inden-2-yl}-ethyl)-amine 10-1 as the TFA salt, 15% yield. (MH⁺=375.1)

In a similar manner dimethyl-(2-{3-[1-(5-methyl-thiazol-2-yl)-ethyl]-1H-inden-2-yl}-ethyl)-amine 10-2 was prepared from compound 11b. (MH⁺=313)

The following compounds were made according to this procedure:

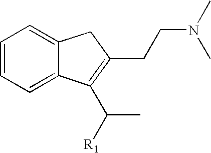

| No. | R₁ | MW | MH⁺ | t_{R} (method 2) |
|---|---|---|---|---|
| 10-1 | 5-phenyl-thiazol-2-yl | 374.55 | 375.1 | 5.971 |
| 10-2 | 5-methyl-thiazol-2-yl | 312.48 | 313.0 | 4.570 |

### Example 11

### DIMETHYL-(2-{3-[1-(5-METHYL-OXAZOL-2-YL)-ETHYL]-1H-INDEN-2-yl}-ETHYL)-AMINE

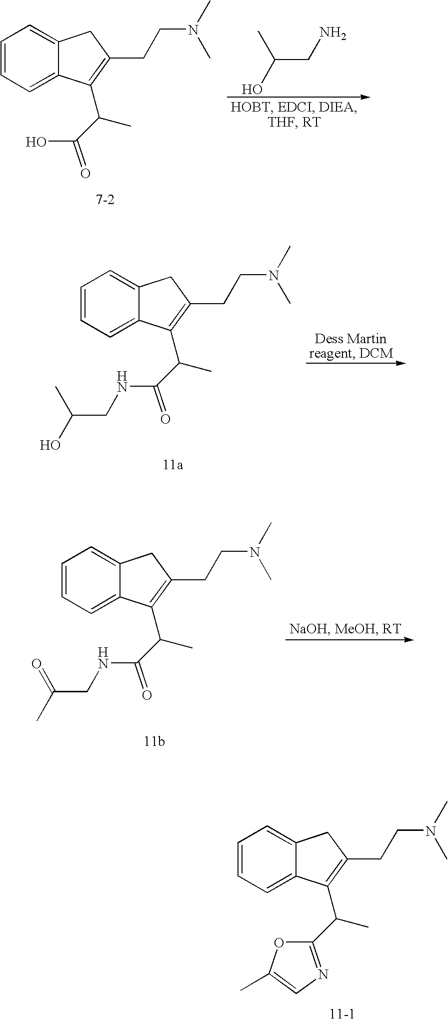

To a solution of compound 7-2 (0.2 g, 0.77 mmol) in THF (2 mL) was added HOBT (0.158 g, 1.16 mmol), EDCI (0.22 g, 1.16 mmol), DIEA (0.27 mL, 1.54 mmol) and DL-1-amino-2-propanol (60 μL, 0.8 mmol). The reaction mixture was stirred at room temperature overnight. The reaction mixture was concentrated in vacuo and purified by flash column chromatography (NH₄OH:MeOH:CH₂Cl₂, 11:5:94) to give compound 11a, 94% yield. (MH⁺=317.2). Compound 11a (70 mg, 0.22 mmol) was dissolved in methylene chloride (1 mL), and Dess Martin reagent (154 mg, 0.38 mmol) was added. The reaction mixture was stirred overnight and quenched with 1N NaOH aqueous solution and brine. The organic solution was concentrated to dryness and purified by flash column chromatography (NH₄OH:MeOH:CH₂Cl₂, 1:5:94) to afford compound 11b, 30% yield. (MH⁺=315.2). A solution of 11b (32 mg, 0.1 mmol) in POCl₃ (0.5 mL) was heated at 125° C. under nitrogen for 2 h. The reaction mixture was cooled, poured onto ice, and rendered basic to pH 7-8 using concentrated NH₄OH. To the ice-cold aqueous layer was added 1 mL of brine, and the mixture was extracted with methylene chloride (3×0.5 mL). The organic layers were combined, dried over MgSO₄ and filtered, and the solvent was removed in vacuo to give a dark oil which was purified by preparative HPLC to afford the TFA salt of compound 11-1, 39% yield. (MH⁺=297.1)

### Example 12

### DIMETHYL-(2-{3-[1-(5-METHYL-4,5-DIHYDRO-OXAZOL-2-YL)-ETHYL]-1H-INDEN-2-YL}-ETHYL)-AMINE

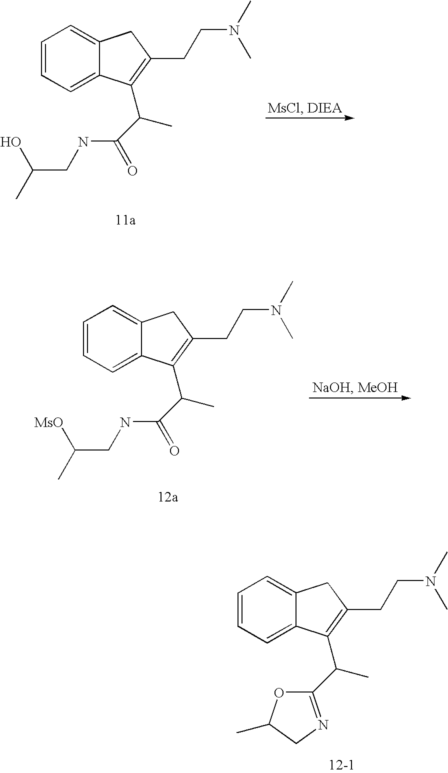

To a solution of 11a (0.1 g, 0.33 mmol) in methylene chloride (2 mL) and DIEA (115 μL, 0.66 mmol), MsCl (39 μL, 0.50 mmol) was added at 0° C. with stirring. The reaction mixture was stirred overnight and concentrated to dryness in vacuo. The residue 12a was dissolved in methanol (2 mL) and 1N NaOH aqueous solution (0.5 mL) was added. The mixture was stirred overnight at room temperature and concentrated to a yellow oil. The yellow oil was purified by flash column chromatography (NH₄OH:MeOH:CH₂Cl₂, 1:5:94) to afford dimethyl-(2-{3-[1-(5-methyl-4,5-dihydro-oxazol-2-yl)-ethyl]-1H-inden-2-yl}-ethyl)-amine 12-1, 69% yield. (MH⁺=299.1)

### Example 13

### (2-{3-[1-(4,5-DIHYDRO-OXAZOL-2-YL)-ETHYL]-1H-INDEN-2-yl}-ETHYL)-DIMETHYL-AMINE

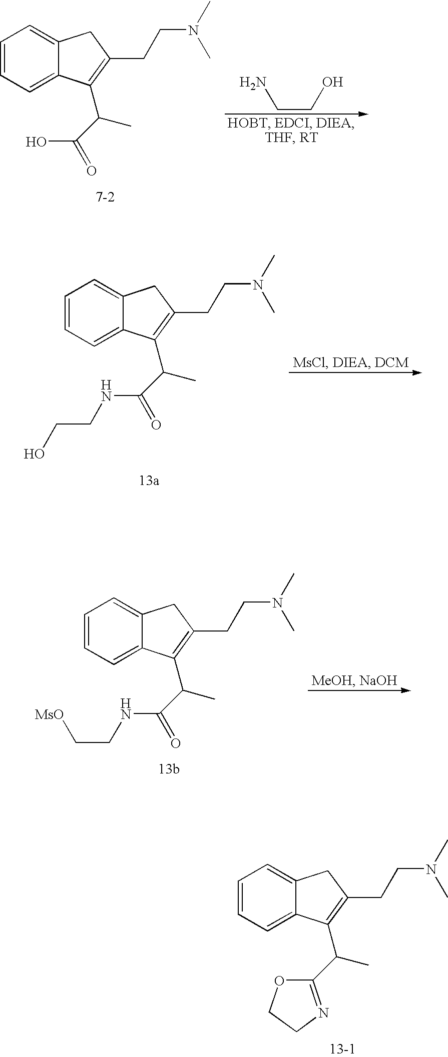

To a solution of 7-2 (0.2 g, 0.77 mmol) in THF (2 mL) was added HOBT (0.158 g, 1.16 mmol), EDCI (0.22 g, 1.16 mmol,) DIEA (27 μL, 1.54 mmol) and ethanolamine (49 μL, 0.8 mmol). The reaction mixture was stirred at room temperature overnight, concentrated in vacuo and purified by flash column chromatography (NH₄OH:MeOH:CH₂C₂, 1:5:94) to afford compound 13a, 94% yield. (MH⁺=303.2). To a solution of 13a (100 mg, 0.33 mmol) in methylene chloride (2 mL) and DIEA (118 μL, 0.66 mmol), MsCl (40 μL, 0.50 mmol) was added at 0° C. with stirring. The reaction mixture was stirred overnight and concentrated to dryness in vacuo. The mesyl compound 13b was dissolved in methanol (2 mL) and 1N NaOH aqueous solution (0.5 mL) was added. The mixture was stirred overnight at room temperature and concentrated to a yellow oil. The yellow oil was purified by flash column chromatography (NH₄OH:MeOH:CH₂Cl₂, 1:5:94) to afford (2-{3-[1-(4,5-dihydro-oxazol-2-yl)-ethyl]-1H-inden-2-yl}-ethyl)-dimethyl-amine 13-1, 71% yield. (MH⁺=285.1)

### Example 14

### DIMETHYL-(2-{3-[1-(5-METHYL-[1,3,4]THIADIAZOL-2-YL)-ETHYL]-1H-INDEN-2-yl}-ETHYL)-AMINE

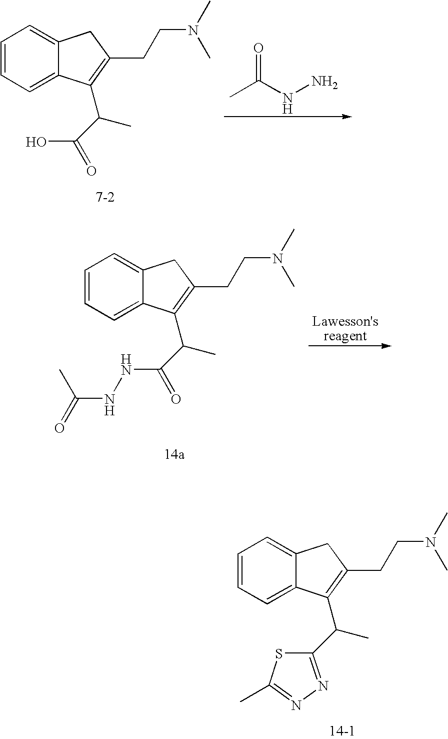

To a solution of 7-2 (0.1 g, 0.38 mmol) in dichloromethane (10 mL), acetic acid hydrazide (72 mg, 0.965 mmol), EDCI (0.111 g, 0.58 mmol), HOBT (79 mg, 0.59 mmol) and DIEA (134 μL, 0.77 mmol) were added. The reaction mixture was stirred at room temperature overnight. Additional dichloromethane was added and the mixture was washed with water. The organic layer was dried and concentrated in vacuo to give 14a. The residue was dissolved in toluene (1 mL) and Lawesson's reagent (0.32 g, 0.78 mmol) was added. The mixture was refluxed overnight at room temperature.

After addition of 3N HCl the reaction mixture was washed with diethyl ether. The aqueous solution was neutralized with NH₄OH and extracted with dichloromethane. The combined organic layers were dried (MgSO₄), filtered and concentrated in vacuo to give 20 mg of dimethyl-(2-{3-[1-(5-methyl-[1,3,4]thiadiazol-2-yl)-ethyl]-1H-inden-2-yl}-ethyl)-amine 14-1 (17% yield). (MH⁺=314).

The following compounds were made according to this procedure:

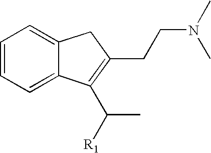

| No. | R₁ | MW | MH⁺ | t_{R} (method 2) |
|---|---|---|---|---|
| 14-1 | 5-methyl-[1,3,4]thiadiazol-2-yl | 313.47 | 314 | 3.654 |
| 14-2 | 5-phenyl-[1,3,4]thiadiazol-2-yl | 375.54 | 376 | 5.173 |

### Example 15

### METHYL-{2-[3-(1-PYRAZIN -2-YL-ETHYL)-1H-INDEN-2-YL]-ETHYL}-AMINE

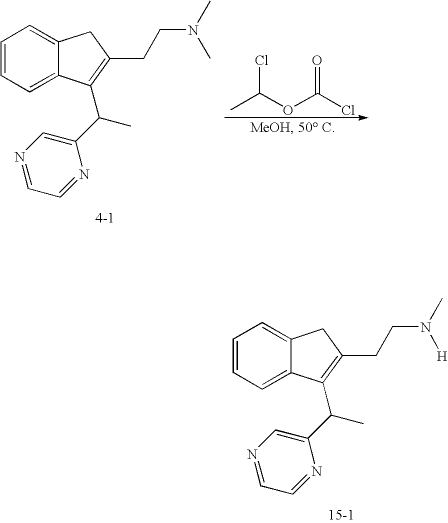

To a solution of dimethyl-{2-[3-(1-pyrazin-2-yl-ethyl)-1H-inden-2-yl]-ethyl}-amine 4-1 (180 mg, 0.61 mmol) in dichloroethane, α-chloroethylformate (86 μL, 0.76 mmol) was added at 0° C. After stirring for 15 min at rt and 50 min at 90° C., the mixture was concentrated in vacuo, taken up in dichloromethane, washed with water (2×), HCl (1N), water, dried (Na₂SO₄) and concentrated in vacuo. The residue was dissolved in MeOH and heated at 90° C. for 2 hrs in a sealed tube, concentrated in vacuo and purified by silica gel column chromatography (dichloromethane/MeOH=98/2 up to 92/8) to afford 15-1 (12% yield). (MH+=280).The enantiomers were separated using chiralcel OD-H column chromatography (profile 9) to give methyl-{2-[3-((R)-1-pyrazin-2-yl-ethyl)-1H-inden-2-yl]-ethyl}-amine 15-2 and methyl-{2-[3-((S)-1-pyrazin-2-yl-ethyl)-1H-inden-2-yl]-ethyl}-amine 15-3. Alternatively, instead of dissolving the residue in MeOH, the residue may be taken up in 1N HCl (5 mL), stirred at 40° C. for 30 min, concentrated and purified by preparative HPLC to afford 15-6. (MH⁺=299, t_{R}=4.436 min (2)

The following compounds were made according to this procedure:

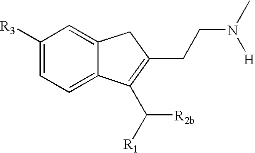

| No. | R₁ | R_{2b} | R₃ | MW | MH⁺ | t_{R} (method) |
|---|---|---|---|---|---|---|
| 15-1 | Pyrazin-2-yl | —CH₃ | H | 279.38 | 280 | 3.82 |
| 15-2 | Pyrazin-2-yl | (R)-CH₃ | H | 279.38 | 280 | 3.699 |
| | | profile 9 | | | | |
| 15-3 | Pyrazin-2-yl | (S)-CH₃ | H | 279.38 | 280 | 3.767 |
| | | profile 9 | | | | |
| 15-4 | Pyrazin-2-yl | H | H | 265.36 | 266 | 3.380 |
| 15-5 | Pyrazin-2-yl | CH₃ | CH₃ | 293.41 | 294 | 4.379 |
| 15-6 | 4-methyl- | CH₃ | H | 298.45 | 299 | 4.436 |
| | thiazol-2-yl | | | | | |

### Example 16

### ETHYL-METHYL-[2-(3-PYRAZIN-2-YL-METHYL-1H-INDEN-2-YL)-ETHEYL]-AMINE

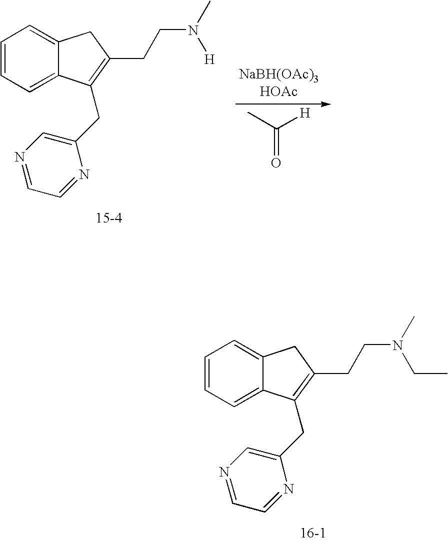

To a solution of methyl-[2-(3-pyrazin-2-ylmethyl-1H-inden-2-yl)-ethyl]-amine 15-4 (0.052 mmol, 15 mg) dissolved in dimethylacetmamide (400 μL), acetaldehyde (20 μL, 0.36 mmol) and sodium triacetoxyborohydride (18 mg, 0.085 mmol) were added. The mixture was stirred overnight at rt, diluted with MeOH, and purified by preparative HPLC to afford 9 mg of ethyl-methyl-[2-(3-pyrazin-2-ylmethyl-1H-inden-2-yl)-ethyl]-amine 16-1. (MH⁺=293.9)

The following compounds were made according to this procedure:

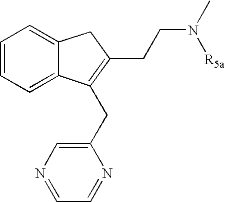

| No. | R₁ | MW | MH⁺ | t_{R} (method 2) |
|---|---|---|---|---|
| 16-1 | —CH₂CH₃ | 293.41 | 293.9 | 3.700 |
| 16-2 | —CH₂CH₂CH₃ | 307.44 | 307.9 | 4.041 |
| 16-3 | —CH₂CH₂OH | 309.41 | 310.1 | 3.377 |

### Example 17

### (2-FLUORO-ETHYL)-METHYL-[2-(3-PYRAZIN-2-ylmethyl-1H-INDEN-2-YL)-ETHYL]-AMINE

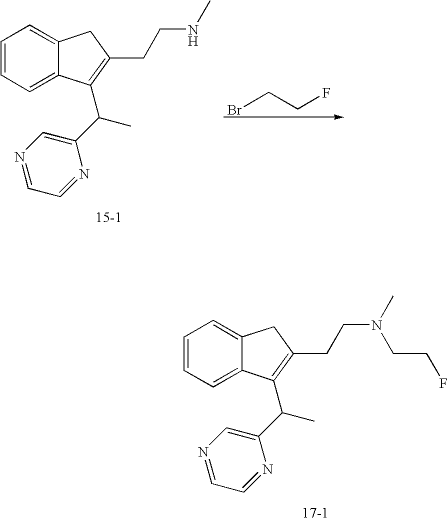

To a solution of methyl-[2-(3-pyrazin-2-ylmethyl-1H-inden-2-yl)-ethyl]-amine 15-1 (37 mg, 0.14 mmol) dissolved in acetone (400 μL), potassium carbonate (31 mg, 0.22 mmol) and 1-bromo-2-fluoro-ethane (9 μL, 0.13 mmol) were added. The mixture was refluxed for 3 hours, additional 1-bromo-2-fluoro-ethane (20 μL, 0.27 mmol) was added and refluxing was continued for 5 hours. The mixture was filtered, concentrated in vacuo, dissolved in MeOH and purified by preparative HPLC to afford 12 mg of (2-fluoro-ethyl)-methyl-[2-(3-pyrazin-2-ylmethyl-1H-inden-2-yl)-ethyl]-amine 17-1. (MH⁺=312.2)

### Example 18

### DIMETHYL-[1-METHYL-2-(3-PYRAZIN-2-YLMETHYL-1H-INDEN-2-YL)-ETHYL]-AMINE

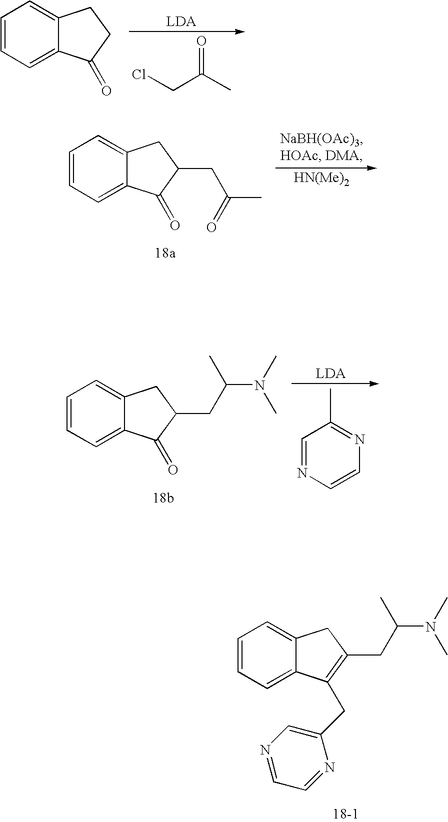

1-Indanone (7.92 g, 60 mmol) dissolved in THF (20 mL) was added dropwise to a solution of LDA (30 mL, 2N solution in heptanes/THF/ethylbenzene, 60 mmol) in THF (200 mL) at −78° C. After stirring at −78° C. for 1 h, chloroacetone (5 mL, 5.8 g, 63 mmol) in THF (20 mL) was added dropwise. The resultant mixture was warmed to rt overnight. The reaction mixture was quenched with saturated NH₄Cl (50 mL) and water (50 mL). The organic layer was separated and the aqueous solution was extracted with EtOAc (200 mL). The combined organic layers were dried (Na₂SO₄), concentrated in vacuo and purified by flash chromatography (eluent:hexanes/EtOAc=10/1) to gave 2-(2-oxo-propyl)-indan-1-one 18a (9.8 g, 86% yield).

Dimethylamine (3 mL, 2M solution in THF, 6 mmol) was added to a solution of (2-oxo-propyl)-indan-1-one 18a (1.128 g, 6 mmol) in DMA (10 mL) and HOAc (0.1 mL). After stirring for 0.5 hr at rt, NaBH(OAc)₃ (1.4 g, 6.6 mmol) was added. After 24 hours, the mixture was diluted with EtOAc and 1N HCl. The acid layer was separated, neutralized with NH₄OH and extracted with EtOAc. The combined organic layers were dried (Na₂SO₄), filtered and concentrated in vacuo to give 2-(2-dimethylamino-propyl)-indan-1-one 18b which was used for the next step without further purification.

To a solution of LDA (1 mL, 2M solution, 2 mmol) in THF (5 mL) at 0° C., methylpyrazine (0.2 mL, 104 mg, 2.2 mmol) was added dropwise. After stirring at 0° C. for 0.5 h, 2-(2-dimethylamino-propyl)-indan-1-one 18b (109 mg, 0.5 mmol) in THF (2 mL) was added dropwise. The mixture was stirred at rt overnight. A small piece of ice was added to quench the reaction. The mixture was extracted with Et₂O, washed with NaHCO₃ aqueous solution, then extracted with 20% HCl (2×5 mL). The combined HCl layers were heated to 100° C. for 1 hour. After cooling to room temperature, the mixture was neutralized with cooled NH₄OH and extracted with EtOAc. The organic layer was dried over Na₂SO₄ and concentrated in vacuo. Purification by HPLC gave dimethyl-[1-methyl-2-(3-pyrazin-2-ylmethyl-1H-inden-2-yl)-ethyl]-amine 18-1 as the TFA salt. (MH⁺=293.9).

Separation by chiral HPLC (profile 8) afforded the (S) and (R) enantiomers, compounds 18-2 and 18-3, respectively.

The following compounds were made according to this procedure:

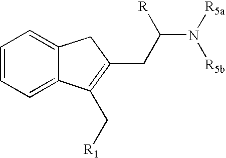

| No. | R₁ | R | —NR₅ₐR_{5b} | MW | MH⁺ | t_{R}(method 2) |
|---|---|---|---|---|---|---|
| 18-1 | Pyrazin- | CH₃ | —N(CH₃)₂ | 293.41 | 293.9 | 3.652 |
| | 2-yl | | | | | |
| 18-2 | Pyrazin | (S)-CH₃ | —N(CH₃)₂ | 293.41 | 293.9 | 3.636 |
| | 2-yl | profile 8 | | | | |
| 18-3 | Pyrazin | (R)-CH₃ | —N(CH₃)₂ | 293.41 | 293.9 | 3.640 |
| | 2-yl | profile 8 | | | | |
| 18-4 | Pyrazin- | CH₃ | Pyrrolidin- | 319.45 | 319.9 | 3.969 |
| | 2-yl | | 1-yl | | | |
| 18-5 | Pyrida- | CH₃ | Pyrrolidin- | 319.45 | 319.9 | 3.646 |
| | zin-3-yl | | 1-yl | | | |

### Example 19

### 3-[5-FLUORO-2-(2-PYRROLIDIN-1-YL-ETHYL)-3H-INDEN-1-YLMETHYL]-PYRIDAZINE

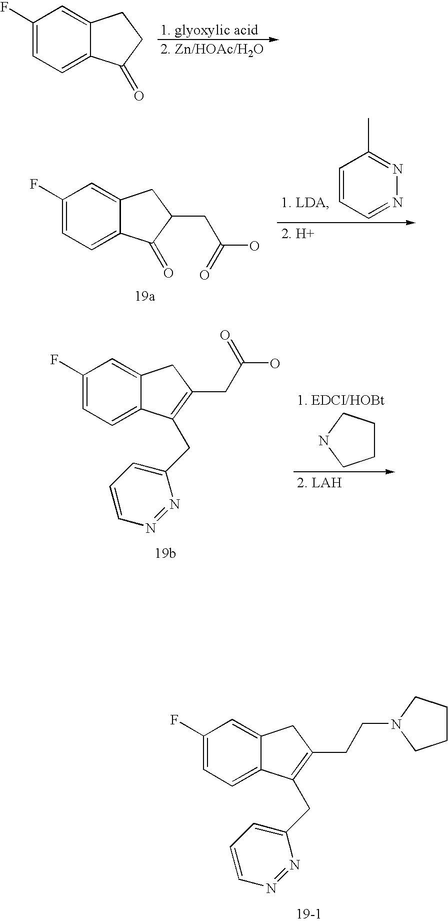

A mixture of 5-fluoro-1-indanone (15 g, 100 mmol), glyoxylic acid (40% in H₂O, 37 g, 200 mmol) and sulfuric acid (98%, 5 mL) in dioxane (100 mL) was refluxed for 5 hrs. After cooling to room temperature, the precipitated solid was filtered and washed with water (20 mL). The filtrate was concentrated in vacuo to generate a solid which was washed with H₂O (100 mL). The combined solids were dissolved in acetic acid (200 mL) then water (80 mL) and zinc dust (7.8 g, 120 mmol) were added. The resultant mixture was stirred at 100° C. for 1 hour and allowed to cool down to room temperature. The excess zinc was filtered off. Water was added to the filtrate and the mixture was extracted with Et₂O (2×200 mL). The organic layers were combined, dried over Na₂SO₄, filtered and concentrated to give (5-fluoro-1-oxo-indan-2-yl)-acetic acid 19a as an oil which solidified upon standing.

Similarly (5-methoxy-1-oxo-indan-2-yl)-acetic acid 19a.1 (MH⁺=220.9) and (1-oxo-indan-2-yl)-acetic acid 19a.2 (MH⁺=190.9) were prepared.

To a solution of LDA (40 mL, 80 mmol, 2N solution) in THF (400 mL) at 0° C., 3-methylpyridazine (8.5 g, 90 mmol) was added dropwise. After stirring for 15 min at 0° C., a solution of (5-fluoro-1-oxo-indan-2-yl)-acetic acid 19a (4.16 g, 20 mmol) in THF (100 mL) was added dropwise. The resultant mixture was stirred for 2 hours at 0° C. and subsequently quenched with NH₄OH (4 mL). The reaction mixture was extracted with 5% NH₄OH (3×20 mL). The combined aqueous extractions were washed with EtOAc (50 mL) and then concentrated to give an oil which was dissolved in 20% HCl (20 mL) and heated to 95° C. for 1 hour. After addition of HOAc (10 mL) the mixture was cooled to room temperature. HOAc was removed under vacuum. The residue was dissolved in water (150 mL) and extracted with EtOAc (3×200 mL). The combined organic layers were washed with brine (150 mL), dried over Na₂SO₄ and concentrated to give (6-fluoro-3-pyridazin-3-ylmethyl-1H-inden-2-yl)-acetic acid 19b as an oil which was used without further purification.

To a solution of (6-fluoro-3-pyridazin-3-ylmethyl-1H-inden-2-yl)-acetic acid 19b (100 mg, 0.35 mmol) in DMF(5 mL), HOBt (71 mg, 0.52 mmol), EDCI (73 mg, 0.38 mmol) and pyrrolidine (60 μL, 0.7 mmol) were added. The mixture was stirred overnight and EtOAc (50 mL) was added. The mixture was washed with 1N HCl (20 mL), 1N NaOH (20 mL) and brine, dried (Na₂SO₄) and concentrated in vacuo to give a residue which was then dissolved in THF (5 mL) and added to a solution of LiAlH₄ (1 mmol in 10 mL THF). The resultant mixture was refluxed for 2 hours. Once cooled to rt, the mixture was dissolved in EtOAc and washed with 1N NaOH. The organic layer was dried (Na₂SO₄), filtered and concentrated in vacuo followed by preparative HPLC purification to give 3-[5-fluoro-2-(2-pyrrolidin-1-yl-ethyl)-3H-inden-1-ylmethyl]-pyridazine 19-1. (MH⁺=324.1)

The following compounds were made according to this procedure:

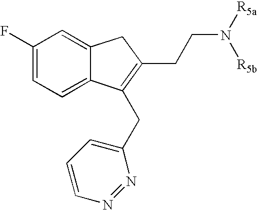

| No. | —NR₅ₐR_{5b} | MW | MH⁺ | t_{R} (method 2) |
|---|---|---|---|---|
| 19-1 | Pyrrolidin-1-yl | 323.41 | 324.1 | 3.616 |
| 19-2 | Morpholin-4-yl | 339.41 | 340.0 | 3.467 |
| 19-3 | 3-(R)-hydroxy-pyrrolidin-1-yl | 339.41 | 340.1 | 3.413 |
| 19-4 | 3-(S)-hydroxy-pyrrolidin-1-yl | 391.41 | 340.1 | 3.449 |

### Example 20

### 3-[2-(2-AZETIDIN-1-YL-ETHYL)-5-FLUORO-3H-INDEN-1-YLMETHYL]-PYRIDAZINE

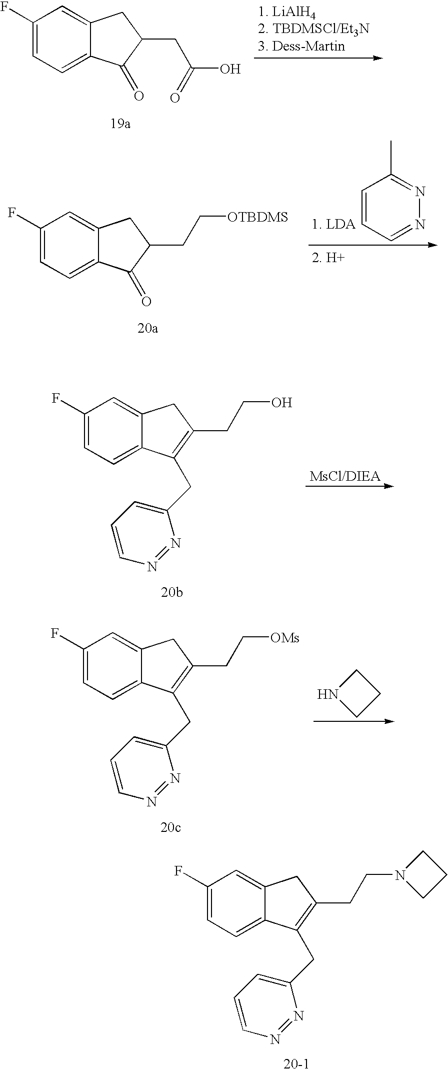

A solution of (5-fluoro-1-oxo-indan-2-yl)-acetic acid 19a (8.5 g, 41 mmol) in THF (100 mL) was added dropwise to a solution of LiAlH₄ (7.6 g, 200 mmol) in THF (200 mL) at 0° C. After stirring at 0° C. for 15 min, the reaction mixture was refluxed for 1 hour. The reaction mixture was cooled to room temperature and carefully quenched by the sequential addition of H₂O, 15% NaOH and H₂O. The mixture was stirred for half an hour, and the solid was filtered off using a celite pad and washed with excess THF. The combined filtrates were dried over Na₂SO₄ and concentrated to afford 5-fluoro-2-(2-hydroxy-ethyl)-indan-1-ol as a white solid (7.67 g, 95% yield, 38 mmol), which was dissolved in dichloromethane (160 mL). Triethylamine (10 mL, 70 mmol), 4-dimethylaminopyridine (484 mg, 3.9 mmol) and t-butyldimethylsilyl chloride (6.47 g, 43 mmol) were added sequentially. After stirring for 2 hrs at room temperature, the reaction mixture was cooled to 0° C. and quenched with saturated NH₄Cl. The organic layer was separated and the aqueous layer was extracted with dichloromethane. The combined organic layers were dried over Na₂SO₄, concentrated and purified by flash chromatography to generate 2-[2-(tert-butyl-dimethyl-silanyloxy)-ethyl]-5-fluoro-indan-1-ol. A solution of this alcohol (3.1 g, 10 mmol) in dichloromethane (50 mL) was added to a solution of Dess-Martin periodinane (5 g, 11.6 mmol) in dichloromethane (100 mL). After stirring at room temperature for 20 min, Et₂O (500 mL) was added followed by 1.3M NaOH (100 mL). The Et₂O layer was separated, washed with 1.3 M NaOH (100 mL) and water (200 mL), dried over Na₂SO₄, concentrated and purified by flash chromatography (hexanes/EtOAc=20/1) to give 2-[2-(tert-butyl-dimethyl-silanyloxy)-ethyl]-5-fluoro-indan-1-one 20a as a colorless oil (2.04 g, 66% yield).

To a solution of LDA (3 mL, 6 mmol) in THF (40 mL) at 0° C., 3-methylpyridazine (658 mg, 7 mmol) was added dropwise. After stirring at 0° C. for 0.5 hr, 2-[2-(tert-butyl-dimethyl-silanyloxy)-ethyl]-5-fluoro-indan-1-one 20a (616 mg, 2 mmol) in THF (5 mL) was added dropwise. After stirring at 0° C. for 2 hours, the reaction was quenched with cold water (50 mL). The organic layer was separated and the aqueous solution was extracted with EtOAc (50 mL). The combined organic layers were washed with brine, concentrated and the residue was dissolved in 20% HCl (5 mL). The mixture was stirred overnight then made basic with NaOH and extracted with EtOAc. The organic extractions were washed with brine, dried over Na₂SO₄, concentrated and purified by flash chromatography (EtOAc up to EtOAc/MeOH=95/5) to give 216 mg of 2-(6-fluoro-3-pyridazin-3-ylmethyl-1H-inden-2-yl)-ethanol 20b (40% yield).

To a solution of 2-(6-fluoro-3-pyridazin-3-ylmethyl-1H-inden-2-yl)-ethanol 20b (27 mg, 0.1 mmol) in dichloromethane (5 mL), DIEA (0.03 mL, 26 mg, 0.2 mmol) and MsCl (0.15 mmol) were added. The resultant mixture was stirred at room temperature overnight. Saturated NaHCO₃ (2 mL) was added to quench the reaction. The mixture was partition end between H₂O (15 mL) and dichloromethane (15 mL). The organic layer was separated, dried over Na₂SO₄ and concentrated to give 20c as a black oil, which was dissolved in iPrOH (2 mL). DIEA (0.3 mmol) and azetidine (0.2 mmol) were added and the mixture was heated at 80° C. overnight, concentrated and purified by preparative HPLC to afford 3-[2-(2-azetidin-1-yl-ethyl)-5-fluoro-3H-inden-1-ylmethyl]-pyridazine 20-1 in 80% yield.

### Example 21

### 2-{1-[2-(2-AZETIDIN-1-YL-ETHYL-5-METHOXY-3H-INDEN-1-YL]-ETHYL}-PYRARAZINE

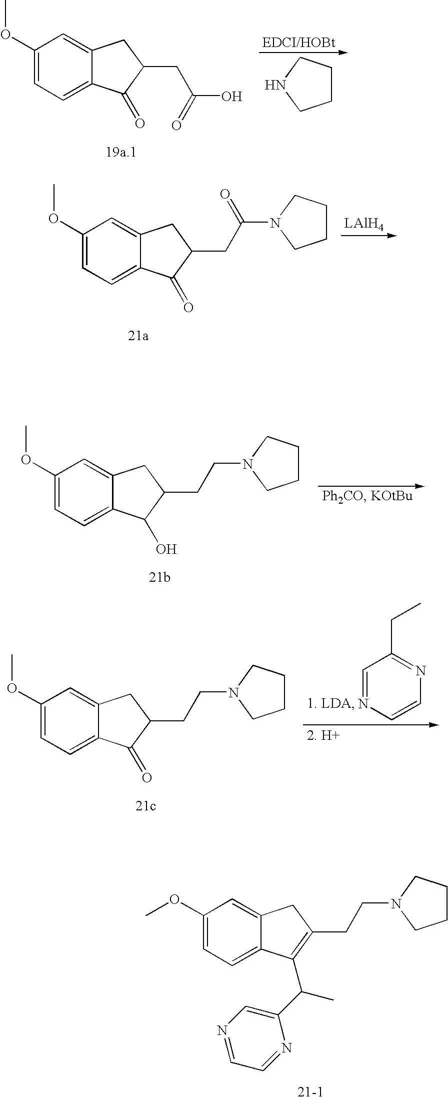

A mixture of(5-methoxy-1-oxo-indan-2-yl)-acetic acid 19a.1 (1 g,4.5 mmol), EDCI (949 mg, 5 mmol), HOBt (1.5 g, 11.3 mmol) and DIEA (1.7 mL, 9 mmol) in 20 mL dichloromethane was stirred at room temperature for 30 min. Pyrrolidine (483 mg, 6.8 mmol) was added and the resulting reaction mixture was stirred at room temperature for 2 hr. Water was added and the organic layer separated, dried over MgSO₄, filtered and concentrated to afford 5-methoxy-2-(2-oxo-2-pyrrolidin-1-yl-ethyl)-indan-1-one 21a in 61% yield. (MH⁺=274)

A solution of 5-methoxy-2-(2-oxo-2-pyrrolidin-1-yl-ethyl)-indan-1-one 21a (700 mg, 2.56 mmol) in 5 mL of THF and 2 mL of dichloromethane was added dropwise to a mixture of LiAlH₄ (150 mg, 3.9 mmol) in 5 mL THF at 0° C. The resulting reaction mixture was stirred for 20 min and then refluxed for 3 hrs. The reaction mixture was cooled to room temperature and carefully quenched by the sequential addition of H₂O, 15% NaOH and H₂O. The mixture was stirred for half an hour, and the solid was filtered off using a celite pad and washed with excess THF. The combined filtrates were dried over MgSO₄ and concentrated to afford 5-methoxy-2-(2-pyrrolidin-1-yl-ethyl)-indan-1-ol 21b as a white solid (421 mg, 63% yield). (MH⁺=262.1)

A mixture of 5-methoxy-2-(2-pyrrolidin-1-yl-ethyl)-indan-1-ol 21b (400 mg, 1.5 mmol), diphenylmethanone (1.4 g, 1.5 mmol) and KOtBu (421 mg, 3.8 mmol) in toluene (10 mL) was heated at 120° C. under N₂ atmosphere for 3 hrs. The cooled mixture was poured into ice (10 g) and extracted with 10% HCl until the HCl solution was colorless. The combined acid extracts were washed with EtOAc and added dropwise with stirring into 28% NH₄OH (10 mL) and ice. This basic solution was extracted with EtOAc (3×10 mL). The combined organic layers were washed with brine, dried over MgSO₄ and concentrated to give 5-methoxy-2-(2-pyrrolidin-1-yl-ethyl)-indan-1-one 21c as an oil (240 mg,62% yield). (MH⁺=260)

Alternatively the oxidation from the substituted indan-1-ol to the substituted indan-1-one may use chromium (VI) oxide as reagent. -A mixture of pyridine (1.5 mL) and dichloromethane (25 mL) was stirred at 0° C. for 15 min. Chromium (VI) oxide (900 mg, 9 mmol) was added and the mixture was stirred for an additional 15 min at 0° C., allowed to warm to room temperature and stirred for an additional hour at room temperature. A solution of 2-[2-(3,3-difluoro-pyrrolidin-1-yl)-ethyl]-indan-1-ol (405 mg, 1.5 mmol) was added and stirring continued for 15 min. After decanting, the dichloromethane layer was washed with water and dried over MgSO₄. Upon concentration 301 mg of the 2-[2-(3,3-difluoro-pyrrolidin-1-yl)-ethyl]-indan-1-one was obtained as an oil (76%) and used in the next step without further purification. (MH⁺=266)

A solution of 2-ethylpyrazine (100 mg, 0.9 mmol) in THF (2 mL) was added dropwise to a mixture of LDA (0.41 mL, 0.82 mmol, 2M) in THF (3 mL) at 0° C. A solution of 5-methoxy-2-(2-pyrrolidin-1-yl-ethyl)-indan-1-one 21c (100 mg, 0.41 mmol) in THF (2 mL) was added dropwise and the resulting mixture was stirred at 0° C. for 1 hr. Water (10 mL) was added to quench the reaction followed by EtOAc (10 mL) and NH₄OH (10 mL). The organic layer was separated and the aqueous layer was extracted with EtOAc (3×3 mL). The combined organic layers were concentrated and the residue was dissolved in 4 mL of 20% HCl and the resulting mixture was stirred at room temperature for 2 hours. The mixture was then poured onto 10 g of ice and 4 mL of NH₄OH was added dropwise. The resulting mixture was stirred and allowed to warm to room temperature. The organic layer was separated and the aqueous layer was extracted with EtOAc (3×). The combined organic layers were dried over MgSO₄ and concentrated. Preparative HPLC chromatography afforded 2-{1-[2-(2-azetidin-1-yl-ethyl)-5-methoxy-3H-inden-1-yl]-ethyl}-pyrazine 21-1. (MH⁺=336.1)

The following compounds were made according to this procedure:

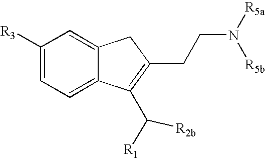

| No | R₃ | R₁ | NR₅ₐR_{5b} | R_{2b} | MW | MH⁺ | t_{R} (method 2) |
|---|---|---|---|---|---|---|---|
| 21-1 | OCH₃ | Pyrazin-2-yl | Azetidin-1-yl | CH₃ | 335.45 | 336.1 | 3.947 |
| 21-2 | OCH₃ | Pyrazin-2-yl | Azetidin-1-yl | (R)CH₃ | 335.45 | 336.1 | 3.941 |
| | | | | Profile 7 | | | |
| 21-3 | OCH₃ | Pyrazin-2-yl | Azetidin-1-yl | (S)_CH₃ | 335.45 | 336.1 | 3.974 |
| | | | | Profile 7 | | | |
| 21-4 | OCH₃ | Pyrazin-2-yl | Pyrrolidin-1-yl | CH₃ | 349.48 | 350.1 | 4.960 |
| 21-5 | OCH₃ | Pyrazin-2-yl | Pyrrolidin-1-yl | H | 335.45 | 336.1 | 3.749 |
| 21-6 | OCH₃ | Pyridazin-3-yl | Pyrrolidin-1-yl | H | 335.45 | 336.1 | 3.500 |
| 21-7 | H | Pyrazin-2-yl | Pyrrolidin-1-yl | H | 305.42 | 306.1 | 3.673 |
| 21-8 | H | Pyridazin-3-yl | Pyrrolidin-1-yl | H | 305.42 | 306.1 | 3.473 |
| 21-9 | OCH₃ | Pyrazin-2-yl | Morpholin-4-yl | H | 351.45 | 352.1 | 3.610 |
| 21-10 | H | Pyrazin-2-yl | Azetidin-1-yl | H | 291.40 | 292.1 | 3.585 |
| 21-11 | H | Pyrazin-2-yl | 3,3-Difluoro- | H | 309.4 | 310 | 3.605 |
| | | | pyrrolidin-1-yl | | | | |
| 21-12 | H | Pyrazin-2-yl | 3-Fluoro- | H | 341.1 | 342.1 | 3.868 |
| | | | azetidin-1-yl | | | | |

### Example 22

### DIMETHYL-(2-{3-[1-(4-METHYL-THIAZOL-2-YL)-ETHYL]-1H-INDEN-2-YL}-ETHYL-AMINE

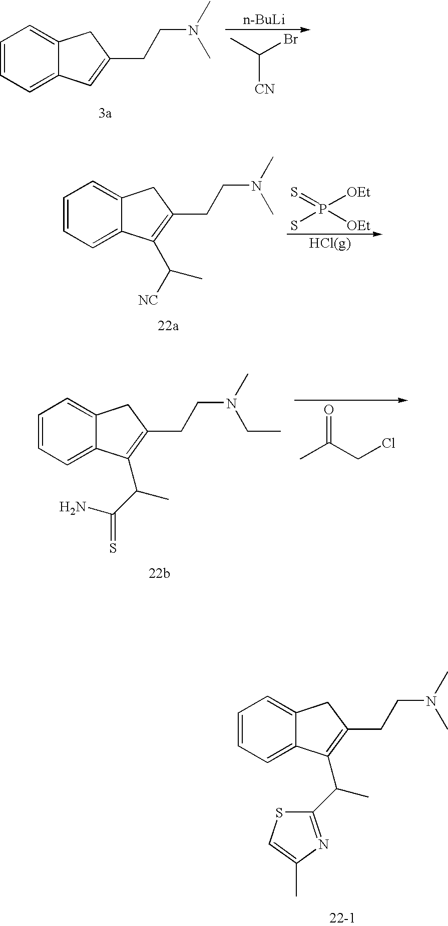

To a solution of [2-(1H-inden-2-yl)-ethyl]-dimethyl-amine 3a (2 g, 10.68 mmol) in anhydrous THF (40 mL) cooled to −78° C., nBuLi (12.8 mmol, 8 mL, 1.6M) was added. After stirring at −78° C. for 1 hr, 2-bromoproprionitrile (10.68 mmol, 0.92 mL) in THF (10 mL) was added. The reaction was mixture warmed to reach room temperature while being stirred overnight. The mixture was concentrated in vacuo. Dichloromethane was added, and the mixture was washed with 1N NaOH and water, dried over MgSO₄, filtered and concentrated in vacuo to afford 2.08 g of 2-[2-(2-dimethylamino-ethyl)-3H-inden-1-yl]-propionitrile 22a which was used in the next step without further purification (83% yield). (MH⁺=241, t_{R}=3.675 (2))

To a solution of 2-[2-(2-dimethylamino-ethyl)-3H-inden 1-yl]-propionitrile 22a (2 g, 8.33 mmol) in ethylacetate (70 mL), dithiophosphoric acid O,O-diethylester (1.55 g,8.33 mmol) in EtOAc (10 mL) was added. HCl was bubbled through the reaction mixture while stirring. After the internal temperature had stabilized, the reaction was sealed and stirred for 3 days. An additional portion of dithiophosphoric acid O,O-diethylester (6.1 mmol, 1 mL) was added and HCl gas was bubbled through while being stirred overnight. The mixture was concentrated in vacuo, diluted with dichloromethane, washed with water, dried (MgSO₄), filtered and concentrated in vacuo. Silica gel column chromatography (gradient of dichloromethane/MeOH/NH₄OH 99/1/0.2 to dichloromethane/MeOH/NH₄OH 98/2/0.2) afforded 0.72 g of 2-[2-(2-dimethylamino-ethyl)-3H-inden-1-yl]-thiopropionamide 22b (31% yield).

To a solution of 2-[2-(2-dimethylamino-ethyl)-3H-inden-1-yl]-thiopropionamide 22b (0.372 g, 1.36 mmol) in EtOH (10 mL), chloroacetone (0.22 mL, 2.72 mmol) was added. The mixture was stirred at 80° C. overnight. The reaction mixture was concentrated in vacuo and the residue was purified using silica gel column chromatography. (dichloromethane/MeOH/NH₄OH 98/2/0.02 as elutant) to afford 0.174 g of dimethyl-(2-{3-[1-(4-methyl-thiazol-2-yl)-ethyl]-1H-inden-2-yl}-ethyl)-amine 22-1 in 41% yield. (MH⁺=313) Separation by chiral HPLC (Chiralpak-AD-H, profile 6) afforded the R and S enantiomers 22-2 and 22-3.

The following compounds were made according to this procedure:

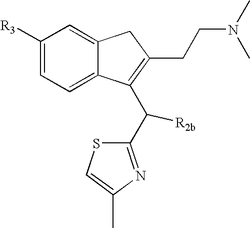

| No | R₃ | R_{2b} | Method | MH⁺ | t_{R} (method 2) |
|---|---|---|---|---|---|
| 22-1 | H | —CH₃ | 312.48 | 313 | 4.363 |
| 22-2 | H | —(R)—CH₃ | 312.48 | 313 | 4.929 |
| | | profile 6 | | | |
| 22-3 | H | —(S)—CH₃ | 312.48 | 313 | 4.875 |
| | | profile 6 | | | |
| 22-4 | OCH₃ | —CH₃ | 342.50 | 342.9 | 4.478 |
| 22-5 | Cl | —(R)—CH₃ | 346.92 | 346.8 | 5.345 |
| | | profile 6 | | | |

### Example 23

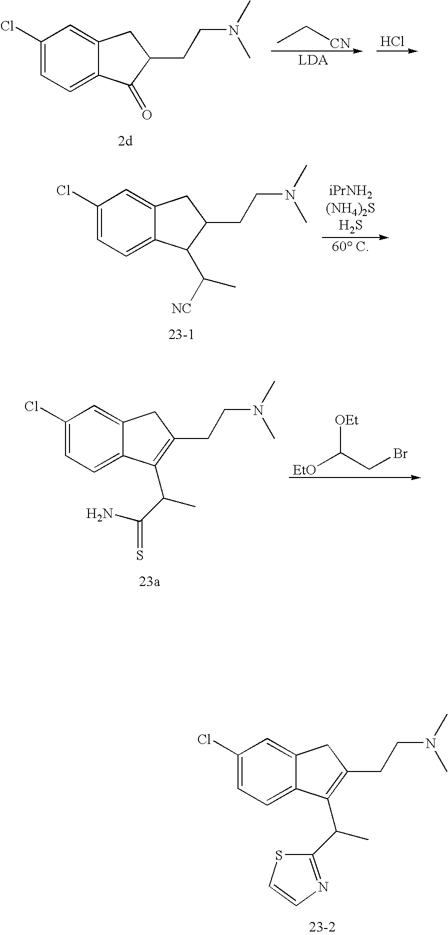

To a cooled (0° C.) solution of propionitrile (0.78 mL, 10.94 mmol) in THF (5 mL), LDA (5.4 mL, 10.94 mmol, 2M) was added. After 30 min at 0° C. a solution of 5-chloro-2-(2-dimethylamino-ethyl)-indan-1-one 2d (0.65 g, 2.73 mmol) in THF (5 mL) was added. After stirring for 30 min, water was added followed by extraction with EtOAc. The combined organic layers were washed with water, dried (MgSO₄) and concentrated in vacuo. 10% HCl (10 mL) was added to the residue and the mixture was stirred at room temperature. The solvent was removed in vacuo and purification by silica gel column chromatography (dichloromethane/MeOH (NH₄OH)=98/2 (0.2%) with gradient up to dichloromethane/MeOH (NH₄OH)=95/5 (0.2%)) afforded 0.27 g of 2-[5-chloro-2-(2-dimethylamino-ethyl)-3H-inden-1-yl]-propionitrile 23-1. (MH⁺=274.8)

To a solution of 2-[5-chloro-2-(2-dimethylamino-ethyl)-3H-inden-1-yl]-propionitrile 23-1 (0.27 g, 1 mmol) in DMA (2 mL), isopropylamine (42 μL) was added. H₂S was bubbled into the reaction mixture which was stirred overnight at 30° C. After preparative HPLC, 0.1 g of 2-[5-chloro-2-(2-dimethylamino-ethyl)-3H-inden-1-yl]-thiopropionamide 23a was isolated.

To a solution 23a in a mixture of EtOH (1 mL) and acetone (1 mL), 2-bromo-1,1-diethoxy-ethane (78 μL, 0.52 mmol) was added along with 2 drops of conc. sulfuric acid. The mixture was stirred overnight at 80° C. and purified by preparative HPLC to generate {2-[6-chloro-3-(1-thiazol-2-yl-ethyl)-1H-inden-2-yl]-ethyl}-dimethyl-amine (52 mg). Both the R and S enantiomers, 23-2 and 23-3 were isolated after chiral HPLC separation.

The following compounds were made according to this procedure:

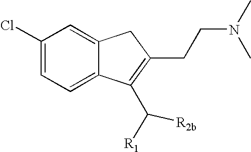

| No. | R₁ | R_{2b} | MW | MH⁺ | t_{R} (method 2) |
|---|---|---|---|---|---|
| 23-1 | —CN | —CH₃ | 274.79 | 274.8 | 4.587 |
| 23-2 | Thiazol-2-yl | —(R)—CH₃ | 332.90 | 332.8 | 4.930 |
| | | profile 6 | | | |
| 23-3 | Thiazol-2-yl | —(S)—CH₃ | 332.90 | 332.8 | 4.910 |
| | | profile 6 | | | |

### Example 24

### 2-(2-DIMETHYLAMINO-ETHYL)-1-PYRAZIN-2-YLMETHYL-3H-INDEN-5-OL

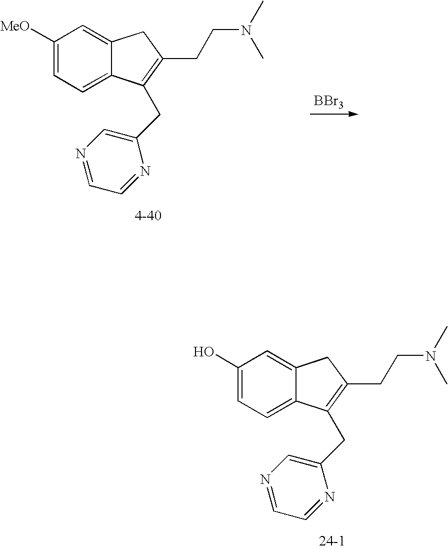

A solution of [2-(6-methoxy-3-pyrazin-2-ylmethyl-1H-inden-2-yl)-ethyl]-dimethyl-amine 4-40 (0.5 g, 1.61 mmol) in CHCl₃ (8 mL) was added dropwise to a solution of BBr₃ (3.1 mL, 32.3 mmol) in CHCl₃ (3 mL) at rt. The resulting reaction mixture was stirred at rt for 2.5 hrs. The reaction mixture was quenched with 50 mL of dilute NH₄OH (ice cold) and then stirred at 0° C. for 15 min. The layers were separated and the organic phase was extracted with 40 mL of 1 M NaOH. The aqueous layers were combined and then concentrated to about 25 mL. The pH of the concentrate was adjusted to ˜10 with NaOH. The solution was saturated with NaCl and then extracted with dichloromethane (4×12 mL). The combined organic layers were dried (MgSO₄), filtered and concentrated in vacuo to give a dark brown oil (418 mg), which was purified by preparative HPLC to afford the TFA salt of 2-(2-dimethylamino-ethyl)-1-pyrazin-2-ylmethyl-3H-inden-5-ol 24-1 (378 mg).

### Example 25

### {2-[5-METHOXY-3-(1-THIAZOL-2-YL-ETHYL)-1H-INDEN-2-yl]-ENTHYL}-DIMETHYLAMINE

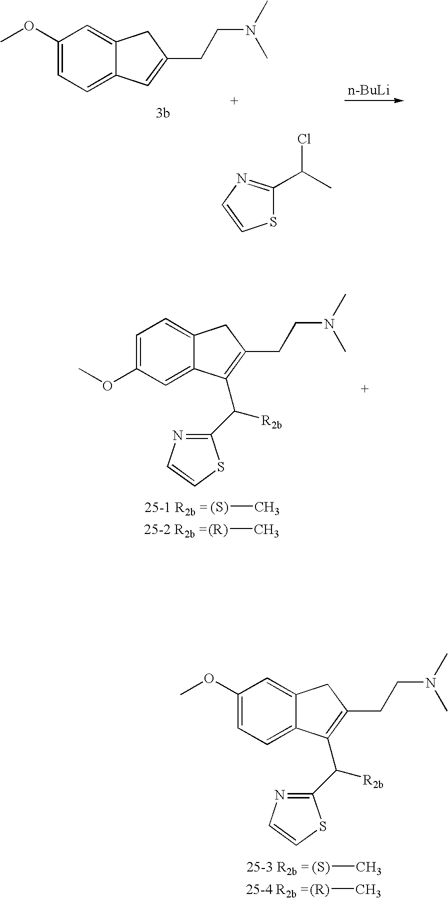

To a solution of 3b (0.148 g, 0.68 mmol) in THF (2 mL) at −78° C., n-Butyllithium (0.44 mL, 0.71 mmol, 1.6M in hexanes) was added. After stirring at −78° C. for 1 hour, 2-(1-chloro-ethyl)-thiazole (0.12 mL, 0.82 mmol) was added. The resultant mixture was warmed to room temperature overnight. Saturated NaHCO₃ (6 mL) and Et₂O (6 mL) were added. The organic layer was isolated and dried over Na₂SO₄ and concentrated to give an oil, which was purified by HPLC to give {2-[5-methoxy-3-(1-thiazol-2-yl-ethyl)-1H-inden-2-yl]-ethyl}-dimethyl-amine and {2-[5-methoxy-3-(1-thiazol-2-yl-ethyl)-1H-inden-2-yl]-ethyl}-dimethyl-amine as TFA salts (20% yield). The enantiomers were separated by chiral HPLC (profile 6) to give 25-1 (3.5 mg, 1.7% yield, MH⁺'329, t_{R}=4.338 min); 25-2 (4.0 mg, 1.8% yield, MH⁺=329, t_{R}=4.246 min); 25-3 (3.1 mg, 1.4% yield, MH⁺329, t_{R}=4.325 min; and 25-4 (3.2 mg, 1.4% yield, MH⁺=329, t_{R}=4.310 min).

2-(1-Chloro-ethyl)-thiazole was prepared as follows: To a cooled solution (0° C.) of 1-thiazol-2-yl-ethanone (10 g, 78.6 mmol) in anhydrous THF (200 mL), LiAlH₄ (3.6 g, 94.4 mmol) was slowly added. After stirring for 30 min, water was added to quench the reaction. THF was removed under vacuum and dichloromethane was added. The mixture was washed with 1N NaOH and water and concentrated in vacuo to yield 0.86 g of 1-thiazol-2-yl-ethanol, which was dissolved in dichloromethane (10 mL). MsCl (0.52 mL, 6.64 mmol) and DIEA (1.74 mL, 9.96 mmol) were added and the reaction mixture was stirred overnight at room temperature. Additional aliquots of MsCl (0.52 mL, 6.64 mmol) and DIEA (0.8 mL, 4.55 mmol) were added and stirring was continued for 16 hours. Additional aliquots were added and the mixture stirred overnight. The solvent was removed in vacuo and the residue purified through silica gel column chromatography (eluent: Hexanes/EtOAc=6/1) to give 0.45 g of 2-(1-chloro-ethyl)-thiazole.

### Example 26

### HUMAN HISTAMINE H₁ RECEPTOR BINDING ASSAY

Compounds of the present invention may be evaluated for binding to the histamine H₁ receptor by a standard binding assay. Crude membranes are prepared from CHO cells transfected with human H₁ receptor expression construct by resuspending cells in lysis buffer (50 mM Tris-HCl pH 7.4, 5 mM EDTA, 10 mM MgCl₂ and disrupting under N₂ at a pressure of 900 psi (Parr Cell disruption bomb, cat.4639) for 30 min on ice followed by differential centrifugation. The resulting crude membrane pellet is resuspended in assay buffer (50 mM Tris HCl pH 7.4, 100 mM NaCl, 2 mM MgCl₂). Membrane protein concentration is adjusted to 1 mg/ml and aliquots were stored at −80° C. An aliquot of membranes (10-20 μg of protein) is incubated for 90 min with 1.5 nM [pyridinyl-5-³H]Pyrilamine (˜30 Ci/mmol, Amersham TRK608) in the presence of varying concentrations of competing ligand. Non-specific binding is determined in the presence of excess (1 μM) doxepin. Bound and free ligand are separated by rapid vacuum filtration using a Packard 96-well cell harvester onto UniFilter GF/C filter plates (PerkinElmer) that has been pretreated with 1% polyethyleneimine. The filter plates are then washed with 600 μl phosphate buffered saline containing 0.01% (v/v) Triton-X100. Bound radioligand is determined by scintillation counting using a TopCount-NXT (Packard). Binding data is analyzed by nonlinear, least-squares curve fitting algorithms using GraphPad Prism (GraphPad Software, Inc. San Diego, Calif.) or ActivityBase (IDBS, Guildford, Surrey, UK). Kᵢ values are calculated from IC₅₀ values using the Cheng-Prusoff equation (Biochem. Pharm. 22: 3099-3108, 1973).

Compounds of this invention generally have a Kᵢ of less than 10 μM, typically less than 1 μM, and preferably less than 250 nM. The following compounds have a Kᵢ of 250 nM or less:

4-1, 4-2, 4-3, 4-4, 4-5, 4-6, 4-7, 4-9, 4-10, 4-11, 4-12, 4-13, 4-14,
4-15, 4-16, 4-17, 4-18, 4-19, 4-20, 4-21, 4-22, 4-23, 4-24, 4-25,
4-26, 4-27, 4-28, 4-29, 4-30, 4-32, 4-33, 4-34, 4-35, 4-36, 4-37,
4-38, 4-39, 4-40, 4-41, 4-42, 4-43, 4-45, 4-46, 4-47
5-1, 5-2, 5-3, 5-4, 5-5, 5-6, 5-7, 5-9, 5-10, 5-11
6-1, 6-2
7-1, 7-3
8-1, 8-2
9-1
10-1, 10-2
11-1
12-1
13-1
14-1, 14-2
15-1, 15-2, 15-4, 15-5, 15-6
16-1, 16-2, 16-3
17-1
18-1, 18-2, 18-3, 18-4, 18-5
19-1, 19-2, 19-3, 19-4
20-1
21-1, 21-2, 21-4, 21-5, 21-6, 21-7, 21-8, 21-9, 21-10, 21-11, 21-12
22-1, 22-2, 22-3, 22-4, 22-5
23-1, 23-2, 23-3
25-2, 25-4

### Example 27

### EEG STUDIES

Adult, male Wistar rats (Charles River Laboratories, 275 g) are anesthetized with inhaled isoflurane and restrained in a stereotaxic device. Using aseptic technique, a sterile 6-lead telemetry-based electroencephalographic/electromyographic recording unit (Transdoma DataSciences Incorporated) is attached to the rat. Pairs of electroencephalographic leads are placed onto the dura in the frontal and occipital cortices. The EMG leads are sutured into nuchal trapezoidal muscles. An additional lead attached to the muscle layer serves as a ground. Leads are affixed to the skull with dental acrylic. The leads and the attached transmitter are enclosed into a subcutaneous pocket between the scapulae. Rats recover for 7-14 days prior to study.

Rats are individually housed in standard cages with filter top covers and ad libitum food and water in an isolated room with a 24-hour light (12 hours)/dark (12 hours) cycle and controlled humidity. Rats are placed on their individual telemetry receivers and assess to the recording room is restricted 24 hours prior to the baseline recording. Baseline recordings began 6 hours after lights off, 24 hours prior to dosing.

Recordings are made using DataSciences telemetric receivers and compiled with DataSciences ART-GOLD 2.3 software at a sampling frequency of 100 Hz. Recordings from one pair of bilateral EEG leads and from the EMG leads are used to divide the vigilance state of rats into Wake and Sleep (NREM and REM). Power spectra of the EEG signal during individual vigilance states are computed from fast-Fourier transforms generated at 512 Hz.

Sedative effects of test compounds are monitored in male Wistar rats after oral administration of test compounds and vehicle control (0.25% methylcellulose). Compounds are administered during the activity portion of the diurnal cycle, 6 hours after lights-off.

It will be appreciated that, although specific embodiments of the invention have been described herein for purposes of illustration, various modifications may be made without departing from the spirit and scope of the invention. Accordingly, the invention is not limited except as by the appended claims.

1. A method for treating a condition or disorder, the treatment of which can be effected or facilitated by antagonizing a histamine receptor, comprising administering to a patient in need thereof, an effective amount of a compound having the following structure: 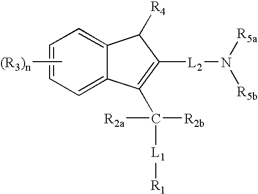
or a stereoisomer, prodrug or pharmaceutically acceptable salt thereof,
wherein:
R₁ is R₁ₐ, R_{1b}, —OR, —CN, —C(═O)R, —OC(═O)R or —C(═O)OR, wherein R is alkyl or substituted alkyl;
R₁ₐ is heterocycle or substituted heterocycle, with the proviso that R₁ₐ is not pyridinyl or substituted pyridinyl;
R_{1b} is bicyclic carbocycle or substituted bicyclic carbocycle;
L₁ is a bond or L₂;
L₂ is alkanediyl or substituted alkanediyl;
R₂ₐ and R_{2b} are the same or different and are independently hydrogen, alkyl or substituted alkyl;
R₃ is, at each occurrence, the same or different and independently alkyl, —OR, —SR, —CN, —CF₃ or halogen, wherein R is alkyl or substituted alkyl;
R₄ is hydrogen or alkyl;
R₅ₐ and R_{5b} are the same or different and independently hydrogen, alkyl or substituted alkyl, or R₅ₐ and R_{5b} together with the nitrogen to which they are attached form a heterocycle or substituted heterocycle; and
n is 0, 1 or 2 and represents the number of R₃ groups.

2. A method for treating insomnia, inducing sleep, or inducing sedation or hypnosis in a patient in need thereof, comprising administering to the patient an effective amount of a compound having the following structure: 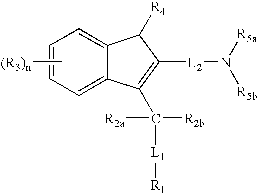
or a stereoisomer, prodrug or pharmaceutically acceptable salt thereof,
wherein:
R₁ₐ is R₁ₐ, R_{1b}, —OR, —CN, —C(═O)R, —OC(═O)R or —C(═O)OR, wherein R is alkyl or substituted alkyl;
R₁ₐ is heterocycle or substituted heterocycle, with the proviso that R₁ₐ is not pyridinyl or substituted pyridinyl;
R_{1b} is bicyclic carbocycle or substituted bicyclic carbocycle;
L₁ is a bond or L₂;
L₂ is alkanediyl or substituted alkanediyl;
R₂ₐ and R_{2b} are the same or different and are independently hydrogen, alkyl or substituted alkyl;
R₃ is, at each occurrence, the same or different and independently alkyl, —OR, —SR, —CN, —CF₃ or halogen, wherein R is alkyl or substituted alkyl;
R₄ is hydrogen or alkyl;
R₅ₐ and R_{5b} are the same or different and independently hydrogen, alkyl or substituted alkyl, or R₅ₐ and R_{5b} together with the nitrogen to which they are attached form a heterocycle or substituted heterocycle; and
n is 0, 1 or 2 and represents the number of R₃ groups.

3. The method of claim 2 wherein L₁ is a bond and L₂ is ethylenediyl.

4. The method of claim 3 wherein R₄ is hydrogen and R₅ₐ and R_{5b} are the same or different and independently alkyl.

5. The method of claim 4 wherein R₁ is R₁ₐ.

6. The method of claim 5 wherein R₁ₐ is pyrazinyl, substituted pyrazinyl, pyridazinyl or substituted pyridazinyl.

7. The method of claim 1 wherein L₁ is a bond and L₂ is ethylenediyl.

8. The method of claim 2 wherein the compound is:
[2-(6-fluoro-3-pyrazin-2-ylmethyl-1H-inden-2-yl)-ethyl]-dimethyl-amine;
dimethyl-[2-(3-pyrazin-2-ylmethyl-1H-inden-2-yl)-ethyl]-amine;
[2-(6-fluoro-3-pyridazin-3-ylmethyl-1H-inden-2-yl)-ethyl]-dimethyl-amine;
{2-[6-fluoro-3-(3-methoxy-pyrazin-2-ylmethyl)-1H-inden-2-yl]-ethyl}-dimethyl-amine;
[2-(6-chloro-3-pyridazin-3-ylmethyl-1H-inden-2-yl)-ethyl]-dimethyl-amine; or
dimethyl-[2-(6-methyl-3-pyridazin-3-ylmethyl-1H-inden-2-yl)-ethyl]-amine.

9. The method of claim 2 wherein the compound is:
dimethyl-{2-[3-(1-pyrazin-2-yl-ethyl)-1H-inden-2-yl]-ethyl}-amine;
(2-{6-fluoro-3-[1-(3-methoxy-pyrazin-2-yl)-ethyl]-1H-inden-2-yl}-ethyl)-dimethyl-amine;
(2-{6-chloro-3-[1-(3-methoxy-pyrazin-2-yl)-ethyl]-1H-inden-2-yl}-ethyl)-dimethyl-amine;
{2-[3-(2-methoxy-1-methyl-ethyl)-1H-inden-2-yl]-ethyl}-dimethyl-amine;
dimethyl-{2-[3-(1-pyrazin-2-yl-ethyl)-1H-inden-2-yl]-ethyl}-amine;
dimethyl-{2-[3-(1-thiazol-2-yl-ethyl)-1H-inden-2-yl]-ethyl}-amine;
{2-[6-methoxy-3-(1-thiazol-2-yl-ethyl)-1H-inden-2-yl]-ethyl}-dimethyl-amine;
{2-[6-methoxy-3-(1-pyrazin-2-yl-ethyl)-1H-inden-2-yl]-ethyl}-dimethyl-amine;
dimethyl-{2-[6-methyl-3-(1-pyrazin-2-yl-ethyl)-1H-inden-2-yl]-ethyl}-amine; or
(2-{3-[1-(3-methoxy-pyrazin-2-yl)-ethyl]-6-methyl-1H-inden-2-yl}-ethyl)-dimethyl-amine.

10. A compound having the following structure: 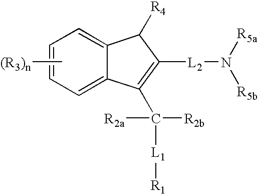
or a stereoisomer, prodrug or pharmaceutically acceptable salt thereof,
wherein:
R₁ is R₁ₐ, R_{1b}, —OR, —C(═O)R, —OC(═O)R or —C(═O)OR, wherein R is alkyl or substituted alkyl;
R₁ₐ is heterocycle or substituted heterocycle, with the proviso that R₁ₐ is not pyridinyl or substituted pyridinyl;
R_{1b} is bicyclic carbocycle or substituted bicyclic carbocycle;
L₁ is a bond or L₂;
L₂ is alkanediyl or substituted alkanediyl;
R₂ₐ and R_{2b} are the same or different and are independently hydrogen, alkyl or substituted alkyl, with the proviso that when R₂ₐ and R_{2b} are both hydrogen, then R₁ is R₁ₐ wherein R₁ₐ is pyrazinyl, substituted pyrazinyl, pyridazinyl, substituted pyridazinyl, triazinyl, or substituted triazinyl;
R₃ is, at each occurrence, the same or different and independently alkyl, —OR, —SR, —CN, —CF₃ or halogen, wherein R is alkyl or substituted alkyl;
R₄ is hydrogen or alkyl;
R₅ₐ and R_{5b} are the same or different and independently hydrogen, alkyl or substituted alkyl, or R₅ₐ and R_{5b} together with the nitrogen to which they are attached form a heterocycle or substituted heterocycle; and
n is 0, 1 or 2 and represents the number of R₃ groups.

11. The compound of claim 10 wherein R₁ is R₁ₐ or R_{1b}.

12. The compound of claim 11 wherein R₁ is R₁ₐ.

13. The compound of claim 12 wherein L₁ is a bond and L₂ is ethylenediyl.

14. The compound of claim 13 wherein R₄ is hydrogen and R₅ₐ and R_{5b} are the same or different and independently alkyl.

15. The compound of claim 14 wherein R₁ₐ is pyrazinyl, substituted pyrazinyl, pyridazinyl or substituted pyridazinyl.

16. The compound of claim 15 wherein R₂ₐ is hydrogen and R_{2b} is methyl.

17. The compound of claim 15 wherein R₂ₐ and R_{2b} are both hydrogen.

18. The compound of claim 12 wherein R₁ₐ is pyrazinyl, substituted pyrazinyl, pyridazinyl or substituted pyridazinyl.

19. The compound of claim 10 wherein R₁ is —OR, —C(═O)R, —OC(═O)R or —C(═O)OR.

20. The compound of claim 19 wherein L₁ is a bond and L₂ is ethylenediyl.

21. The compound of claim 10 wherein R₂ₐ is hydrogen and R_{2b} is alkyl.

22. The compound of claim 10 wherein R₂ₐ and R_{2b} are both hydrogen.

23. The compound of claim 10 wherein R₅ₐ and R_{5b} are the same or different and independently alkyl.

24. The compound of claim 10 wherein n is 0.

25. The compound of claim 10 wherein n is 1.

26. The compound of claim 10 wherein R₄ is hydrogen.

27. The compound of claim 10 wherein R₅ₐ and R_{5b} together with the nitrogen to which they are attached form a heterocyclic ring which is optionally substituted with alkyl or substituted alkyl.

28. The compound of claim 21 wherein L₁ is a bond and L₂ is ethylenediyl.

29. The compound of claim 10, wherein the compound is:
[2-(6-fluoro-3-pyrazin-2-ylmethyl-1H-inden-2-yl)-ethyl]-dimethyl-amine;
dimethyl-[2-(3-pyrazin-2-ylmethyl-1H-inden-2-yl)-ethyl]-amine;
[2-(6-fluoro-3-pyridazin-3-ylmethyl-1H-inden-2-yl)-ethyl]-dimethyl-amine;
{2-[6-fluoro-3-(3-methoxy-pyrazin-2-ylmethyl)-1H-inden-2-yl]-ethyl}-dimethyl-amine;
[2-(6-chloro-3-pyridazin-3-ylmethyl-1H-inden-2-yl)-ethyl]-dimethyl-amine; or
dimethyl-[2-(6-methyl-3-pyridazin-3-ylmethyl-1H-inden-2-yl)-ethyl]-amine.

30. The compound of claim 8, wherein the compound is:
dimethyl-{2-[3-(1-pyrazin-2-yl-ethyl)-1H-inden-2-yl]-ethyl}-amine;
(2-{6-fluoro-3-[1-(3-methoxy-pyrazin-2-yl)-ethyl]-1H-inden-2-yl}-ethyl)-dimethyl-amine;
(2-{6-chloro-3-[1-(3-methoxy-pyrazin-2-yl)-ethyl]-1H-inden-2-yl}-ethyl)-dimethyl-amine;
{2-[3-(2-methoxy-1-methyl-ethyl)-1H-inden-2-yl]-ethyl}-dimethyl-amine;
dimethyl-{2-[3-(1-pyrazin-2-yl-ethyl)-1H-inden-2-yl]-ethyl}-amine;
dimethyl-{2-[3-(1-thiazol-2-yl-ethyl)-1H-inden-2-yl]-ethyl}-amine;
{2-[6-methoxy-3-(1-thiazol-2-yl-ethyl)-1H-inden-2-yl]-ethyl}-dimethyl-amine;
{2-[6-methoxy-3-(1-pyrazin-2-yl-ethyl)-1H-inden-2-yl]-ethyl}-dimethyl-amine;
dimethyl-{2-[6-methyl-3-(1-pyrazin-2-yl-ethyl)-1H-inden-2-yl]-ethyl}-amine; or
(2-{3-[1-(3-methoxy-pyrazin-2-yl)-ethyl]-6-methyl-1H-inden-2-yl}-ethyl)-dimethyl-amine.

31. A pharmaceutical composition comprising a compound of claim 10 and a pharmaceutically acceptable carrier or diluent.

32. A pharmaceutical composition comprising a compound of claim 29 and a pharmaceutically acceptable carrier or diluent.

33. A pharmaceutical composition comprising a compound of claim 30 and a pharmaceutically acceptable carrier or diluent.
